(19) United States
(12) Patent Application Publication
(10) Pub. No.: US 2026/0256143 A1
(43) Pub. Date: Sep. 3, 2026

(54) HERBICIDAL COMPOSITION AND METHOD FOR CONTROLLING WEEDS

(71) Applicant: Enko Chem, Inc., Mystic, CT (US)

(72) Inventors: Neville John ANTHONY, Northborough, MA (US); Karthik PUTTA, Quaker Hill, CT (US); Paolo CASTIGLIONI, Davis, CA (US)

(73) Assignee: Enko Chem, Inc., Mystic, CT (US)

(21) Appl. No.: 19/165,087

(22) PCT Filed: Mar. 13, 2024
(86) PCT No.: PCT/US2024/019775

§ 371 Date: Sep. 12, 2025

Related U.S. Application Data
(60) Provisional application No. 63/452,033, filed on Mar. 14, 2023.

(51) Int. Cl. A01N 43/84; A01N 37/22; A01N 37/40; A01N 39/04; A01N 41/10; A01N 43/707; A01N 43/80; A01N 57/20; A01P 13/00; C07D 265/36

(52) U.S. Cl. CPC A01N 43/84; A01N 37/22; A01N 37/40; A01N 39/04; A01N 41/10; A01N 43/707; A01N 43/80; A01N 57/20; A01P 13/00; C07D 265/36

(57) ABSTRACT

The present application relates to protoporphyrinogen IX oxidase (PPO) herbicidal compositions that include a ring core 2H-benzo[b][1,4]oxazin-3(4H)-one compound having formula (I), substituted at the 6-position with an appropriately substituted phenyl moiety, and at least one compound selected from the group consisting of a second herbicide compound and a herbicide safener.

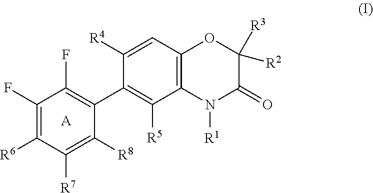

### CROSS-REFERENCE TO RELATED APPLICATIONS

This application claims priority to U.S. Provisional Patent Application No. 63/452,033, filed Mar. 14, 2023, which is incorporated herein by reference in its entirety.

### FIELD

The present invention relates to protoporphyrinogen IX oxidase (PPO) herbicidal compositions that include benzoxazinones having formula (I) and a second herbicide.

### BACKGROUND

Herbicides that inhibit protoporphyrinogen oxidase (hereinafter referred to as Protox or PPO; EC:1.3.3.4), a key enzyme in the biosynthesis of protoporphyrin IX, have been used for selective weed control since the 1960s. PPO catalyzes the last common step in chlorophyll and heme biosynthesis, which is the oxidation of protoporphyrinogen IX to protoporphyrin IX [Matringe M. et al., Protoporphyrinogen oxidase as a molecular target for diphenyl ether herbicides, Biochemistry Journal (1989) 260: 231-235]. Application of PPO-inhibiting herbicides results in the accumulation of protoporphyrinogen IX in the chloroplast and mitochondria, which is believed to leak into the cytosol where it is oxidized by a peroxidase. When exposed to light, protoporphyrin IX causes formation of singlet oxygen in the cytosol and the formation of other reactive oxygen species, which can cause lipid peroxidation and membrane disruption leading to rapid cell death [Lee H. J. et al., Cellular localization of protoporphyrinogen-oxidizing activities of etiolated barley leaves, Plant Physiology (1993) 102: 881].

To date, thousands of PPO inhibitors have been reported in the literature, with about 30 currently used as herbicides to decimate weeds in fields [Hao, G. F., et al., Protoporphyrinogen oxidase inhibitor: an ideal target for herbicide discovery, Chimia (2011) 65, 961-969]. PPO-inhibiting herbicides include many different structural classes of molecules, including diphenyl ethers (e.g. lactofen, acifluorfen, acifluorfen methyl ester, or oxyfluorfen); oxadiazoles (e.g. oxadiazon); cyclic imides [e.g. S-23142, N-(4-chloro-2-fluoro-5-propargyloxyphenyl)-3,4,5,6-tetrahydrophthalimide, chlorophthalim, N-(4 -chlorophenyl)-3,4,5,6-tetrahydrophthalimide)]; phenyl pyrazoles (e.g. TNPP-ethyl, ethyl 2-[1-(2,3,4-trichlorophenyl)-4-nitropyrazolyl-5-oxy]propionate, M&B 39279); pyridine derivatives (e.g. LS 82-556); and phenopylate and its O-phenylpyrrolidino- and piperidinocarbamate analogs (Kramer W., ed., Modern Crop Protection Compounds, 2^{nd} Ed., Vol 1: Herbicides, (2012) Wiley-VCH, Weinheim, Germany). Many of these compounds competitively inhibit the normal reaction catalyzed by the enzyme, apparently acting as substrate analogs.

The herbicidal properties of these known compounds towards harmful plants, however, are not always entirely satisfactory. Herbicide resistant weeds present a serious problem for efficient weed control because such resistant weeds are increasingly widespread and thus weed control by the application of herbicides is no longer effective, causing a huge problem to farmers. Resistance to PPO herbicides has been slow to evolve (about four decades from first commercialization), and to date has been confirmed in 13 weed species [Heap I, The International Survey of Herbicide Resistant Weeds. Available online: http://www.weedscience.org/(October 2019)]. The first weed to evolve resistance to PPO herbicides was waterhemp (Amaranthus tuberculatus) in 2001 [Shoup D. E., et al., Common waterhemp (Amaranthus rudis) resistance to protoporphyrinogen oxidase-inhibiting herbicides Weed Sci. (2003) 51:145-150]. Resistance to PPO herbicides in weedy species has been attributed to target-site mutation in the PPX2 gene. For example, a unique target-site amino acid deletion (Gly₂₁₀) and Arg₉₈Leu substitution confer PPO resistance in waterhemp [Patzoldt W. L., et al., A codon deletion confers resistance to herbicides inhibiting protoporphyrinogen oxidase. Proc. Natl. Acad. Sci. USA (2006) 103:12329-12334] and common ragweed [Rousonelos, et al., Characterization of a common ragweed (Ambrosia artemisiifolia) population resistant to ALS- and PPO-inhibiting herbicides, Weed Sci. (2012) 60:335-344], respectively.

Thus, there is a need for novel methods to effectively control weeds, including herbicide resistant weeds and in particular PPO resistant weeds, which at the same time is tolerated by the useful plants (crops) in question. An object of the present invention is to provide a herbicidal composition and a method for controlling weeds which have an excellent controlling effect on weeds.

### BRIEF SUMMARY

The present invention relates to a herbicidal composition that includes a compound of formula (I), such as 7-fluoro-6-(perfluorophenyl)-4-(prop-2-yn-1-yl)-2H-benzo[b][1,4]oxazin-3(4H)-one, and at least one compound selected from the group consisting of a herbicide compound group B and a safener group C, wherein a weight ratio of the compound of formula (I) to the at least one compound selected from the group consisting of the herbicide group B and the safener group C is 1:0.1 to 1:50. A compound of formula (I) is as follows:

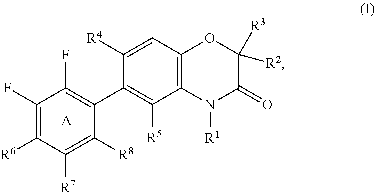

or a salt thereof, wherein:
R¹, R², R³, R⁴, R⁵, R⁶, R⁷, and R⁸ are as described elsewhere herein.

In one embodiment, the compound of formula (I) is 7-fluoro-6-(perfluorophenyl)-4-(prop-2-yn-1-yl)-2H-benzo[b][1,4]oxazin-3(4H)-one (Compound 1), the structure of which is:

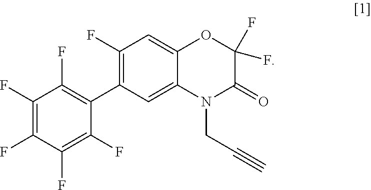 [SMILES: C#CCN1C(=O)C(F)(F)Oc2cc(F)c(-c3c(F)c(F)c(F)c(F)c3F)cc21]

### Definitions

As used herein, the terms “comprises,” “comprising,” “includes,” “including,” “has,” “having,” “contains,” “containing,” “characterized by,” or any other variation thereof, are intended to cover a non-exclusive inclusion, subject to any limitation explicitly indicated. For example, a composition, mixture, process, or method that includes or comprises a list of elements is not necessarily limited to only those elements but may include other elements not expressly listed or inherent to such composition, mixture, process, or method.

The transitional phrase “consisting of” excludes any element, step, or ingredient not specified. If in the claim, such would close the claim to the inclusion of materials other than those recited except for impurities ordinarily associated therewith. When the phrase “consisting of” appears in a clause of the body of a claim, rather than immediately following the preamble, it limits only the element set forth in that clause; other elements are not excluded from the claim as a whole.

Further, unless expressly stated to the contrary, “or” refers to an inclusive ‘or’ and not to an exclusive ‘or.’ For example, a condition A or B is satisfied by any one of the following: A is true (or present) and B is false (or not present), A is false (or not present) and B is true (or present), and both A and B are true (or present).

Also, the indefinite articles “a” and “an” preceding an element or component of the invention are intended to be nonrestrictive regarding the number of instances (i.e. occurrences) of the element or component. Therefore “a” or “an” should be read to include one or at least one, and the singular word form of the element or component also includes the plural unless the number is obviously meant to be singular.

As referred to herein, the term “seedling,” used either alone or in a combination of words means a young plant developing from the embryo of a seed.

As referred to herein, the term “broadleaf,” used either alone or in terms such as “broadleaf weed” means dicot or dicotyledon, a term used to describe a group of angiosperms characterized by embryos having two cotyledons.

The term “acceptable salt” or “salt” when related to a compound of the invention includes cations or anions. Preferred cations are the ions of the alkali metals, preferably of lithium, sodium and potassium, of the alkaline earth metals, preferably of calcium and magnesium, and of the transition metals, preferably of manganese, copper, zinc and iron, further ammonium and substituted ammonium in which one to four hydrogen atoms are replaced by C₁-C₄-alkyl, hydroxy-C₁-C₄-alkyl, C₁-C₄-alkoxy-C₁-C₄-alkyl, hydroxy-C₁-C₄-alkoxy-C₁-C₄-alkyl, phenyl, or benzyl—preferably ammonium, methylammonium, isopropylammonium, dimethylammonium, diethylammonium, diisopropylammonium, trimethylammonium, triethylammonium, tris(isopropyl)ammonium, heptylammonium, dodecylammonium, tetradecylammonium, tetramethylammonium, tetraethylammonium, tetrabutylammonium, 2-hydroxyethylammonium (olamine salt), 2-(2-hydroxyeth-1-oxy)eth- 1-ylammonium (diglycolamine salt), di(2-hydroxyeth-1-yl)ammonium (diolamine salt), tris(2-hydroxyethyl)ammonium (trolamine salt), tris(2-hydroxypropyl)ammonium, benzylthmethylammonium, benzyltriethylammonium, N,N,N-trimethylethanolammonium (choline salt), furthermore phosphonium ions, sulfonium ions, preferably tri(C₁-C₄-alkyl)sulfonium, such as trimethylsulfonium, and sulfoxonium ions, preferably tri(C₁-C₄-alkyl)sulfoxonium, and finally the salts of polybasic amines such as N,N-bis-(3-aminopropyl)methylamine, and diethylenetriamine.

Anions of useful acid addition salts are primarily chloride, bromide, fluoride, iodide, hydrogensulfate, methylsulfate, sulfate, dihydrogenphosphate, hydrogenphosphate, nitrate, bicarbonate, carbonate, hexafluorosilicate, hexafluorophosphate, benzoate, and also the anions of C₁-C₄-alkanoic acids—preferably formate, acetate, propionate, and butyrate.

As used herein, the terms “undesired vegetation” and “harmful plants” are synonyms.

Weed species abbreviations are as follows: AMATU is common waterhemp (Amaranthus rudis), CHEAL is common lambsquarters (Chenopodium album), ABUTH is velvetleaf (Abutilon theophrasti), SETFA is giant foxtail (Setaria faberi), AMAPA is Palmer amaranth (Amaranthus palmeri) AMARE is Redroot Pigweed (Amaranthus retroflexus), KCHSC is kochia (Bassia scoparia), ALOMY is blackgrass (Alopecurus myosuroides), ECHCG is barnyard grass (Echinochloa crus-galli), and DIGSA is large crabgrass (Digitaria sanguinalis).

### DETAILED DESCRIPTION

In one aspect, provided is a herbicidal composition including the compound of formula (I):

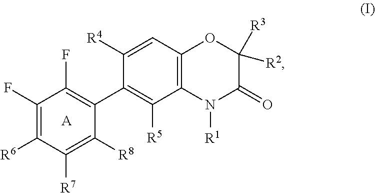

or a salt thereof, wherein:
R¹ is H or C₁₋₄alkyl optionally substituted with Ria, phenyl, or benzyl, wherein each of said alkyl, phenyl or benzyl is optionally substituted with up to 3 F atoms, an OH group, or an OC₁₋₄alkyl group;
R^{1a} is

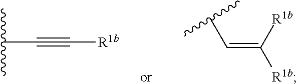

each R^{1b} is, independently, H, C₁₋₄alkyl, or cyclopropyl;
each of R² and R³ is, independently, H, Cl, F, CH₃, or R² and R³ together with the intervening carbon is cyclopropyl;
R⁴ is H, Cl, or F;
R⁵ is H or F;
each of R⁶ and R⁷ is, independently, F, H, C₁₋₂alkyl optionally substituted with OH, alkenyl, OH, OC₁₋₂alkyl, O-cyclopropyl, OCH₂CCH, NHCH₂Ph, N(R^{X})₂, or SCH₃;
R⁸ is H or F;
each R^{x} is, independently, H, CH₃, or C(O)CH₃; and
wherein Ring A contains at least 4 F atom substituents,
wherein at least one compound is additionally included from herbicide group B, and optionally a safener from group C, wherein a weight ratio of the compound of formula (I) to the at least one compound selected from the group consisting of the herbicide compound group B is 1:0.1 to 1:50, and wherein the herbicide from group B is selected from groups B-1 to B-12: B-1, acetolactate synthase inhibitors; B-2, acetyl-CoA carboxylase inhibitors; B-3, protoporphyrinogen IX oxidase inhibitors; B-4, 4-hydrophenylpyruvate dioxygenase inhibitors; B-5, phytoene desaturase inhibitors; B-6, photosystem II inhibitors; B-7, very long chain fatty acid synthesis inhibitors; B-8, microtubule formation inhibitors; B-9, auxin herbicides; B-10, enolpyruvylshikimate 3-phosphate synthase inhibitors; B-11, glutamine synthase inhibitors; and B-12, other herbicides, including agriculturally acceptable salts or derivatives thereof.

In one embodiment, the compound of formula (I) is 7-fluoro-6-(perfluorophenyl)-4-(prop-2-yn-1-yl)-2H-benzo[b][1,4]oxazin-3(4H)-one (Compound 1), the structure of which is:

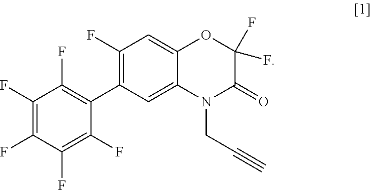 [SMILES: C#CCN1C(=O)C(F)(F)Oc2cc(F)c(-c3c(F)c(F)c(F)c(F)c3F)cc21]

In another embodiment, the B-1 group includes a herbicide from Group B selected from pyrithiobac, pyrithiobac-sodium salt, pyriminobac, pyriminobac-methyl, bispyribac, bispyribac-sodium salt, pyribenzoxim, pyrimisulfan, pyriftalid, triafamone, amidosulfuron, azimsulfuron, bensulfuron, bensulfuron-methyl, chlorimuron, chlorimuron-ethyl, cyclosulfamuron, ethoxysulfuron, flazasulfuron, flucetosulfuron, flupyrsulfuron, flupyrsulfuron-methyl-sodium, foramsulfuron, halosulfuron, halosulfuron-methyl, imazosulfuron, mesosulfuron, mesosulfuron-methyl, metazosulfuron, nicosulfuron, ortho-sulfamuron, oxasulfuron, primisulfuron, primisulfuron-methyl, propyrisulfuron, pyrazosulfuron, pyrazosulfuron-ethyl, rimsulfuron, sulfometuron, sulfometuron-methyl, sulfosulfuron, trifloxysulfuron, trifloxysulfuron-sodium salt, chlorsulfuron, cinosulfuron, ethametsulfuron, ethametsulfuron-methyl, iodosulfuron, iodosulfuron-methyl-sodium, iofensulfuron, iofensulfuron-sodium, metsulfuron, metsulfuron-methyl, prosulfuron, thifensulfuron, thifensulfuron-methyl, triasulfuron, tribenuron, tribenuron-methyl, trifle-sulfuron, triflusulfuron-methyl, tritosulfuron, bencarbazone, flucarbazone, flucarbazone-sodium salt, propoxycarbazone, propoxycarbazone-sodium salt, thiencarbazone, thiencarba-zone-methyl, cloransulam, cloransulam-methyl, diclosulam, florasulam, flumetsulam, metosulam, penoxsulam, pyroxsu-lam, imazamethabenz, imazamethabenz-methyl, imazamox, imazamox-ammonium salt, imazapic, imazapic-ammonium salt, imazapyr, imazapyr-isopropylammonium salt, imazaquin, imazaquin-ammonium, imazethapyr, imazethapyrammonium, and agriculturally acceptable salts and derivatives thereof.

In another embodiment, the B-2 group includes a herbicide from Group B selected from clodinafop, clodinafop-propargyl, cyhalofop, cyhalofop-butyl, diclofop, diclofop-methyl, fenoxaprop, fenoxaprop-ethyl, fenoxaprop-P, fenoxaprop-P-ethyl, fluazifop, fluazifop-butyl, fluazifop-P, fluazifop-P-butyl, haloxyfop, haloxyfop-methyl, haloxyfop-P, haloxyfop-P-methyl, metamifop, propaquizafop, quizalofop, quizalofop-ethyl, quizalofop-P, quizalofop-P-ethyl, alloxydim, clethodim, sethoxydim, tepraloxydim, tralkoxydim, pinoxaden, flufenoximacil, and agriculturally acceptable salts and derivatives thereof.

In another embodiment, the B-3 group includes a herbicide from Group B selected from azafenidin, oxadiazon, oxadiargyl, carfentrazone, carfentrazone-ethyl, saflufenacil, cinidon, cinidon-ethyl, sulfentrazone, pyraclonil, pyraflufen, pyraflufen-ethyl, butafenacil, fluazolate, fluthiacet, fluthiacet-methyl, flufenpyr, flufenpyr-ethyl, flumiclorac, flumiclorac-pentyl, flumioxazin, trifludimoxazin, pentoxazone, oxyfluorfen, acifluorfen, acifluorfen-sodium salt, aclonifen, chlormethoxynil, chlornitrofen, nitrofen, bifenox, fluoroglycofen, fluoroglycofen-ethyl, fomesafen, fomesafen-sodium salt, lactofen, tiafenacil, epyrifenacil, and agriculturally acceptable salts and derivatives thereof.

In another embodiment, the B-4 group includes a herbicide from Group B selected from benzobicyclon, bicyclopyrone, mesotrione, sulcotrione, tefuryltrione, tembotrione, isoxachlortole, isoxaflutole, benzofenap, pyrasulfotole, pyrazolynate, pyrazoxyfen, fenquinotrione, topramezone, tolpyralate, lancotrione, lancotrione-sodium salt, flusulfinam, bipyrazone, fenpyrazone, cypyrafluone, tripyrasulfone, 2-methyl-N-(5-methyl-1,3,4-oxadiazol-2-yl)-3-(methylsulfonyl)-4-(trifluoromethyl)benzamide (CAS Registry Number: 1400904-50-8), 2-chloro-N-(1-methyl-1H-tetrazol-5-yl)-3-(methylthio)-4-(trifluoromethyl)benzamide (CAS Registry Number: 1361139-71-0), 4-(4-fluorophenyl)-6-[(2-hydroxy-6-oxo-1-cyclohexene-1-yl)carbonyl]-2-methyl-1,2,4-triazine-3,5(2H,4H)-dione (CAS Registry Number: 1353870-34-4), and agriculturally acceptable salts and derivatives thereof.

In another embodiment, the B-5 group includes a herbicide from Group B selected from diflufenican, picolinafen, beflubutamid, norflurazon, fluridone, flurochloridone, flurtamone, and agriculturally acceptable salts and derivatives thereof.

In another embodiment, the B-6 group includes a herbicide from Group B selected from ioxynil, ioxynil-octanoate, bentazone, pyridate, bromoxynil, bromoxynil-octanoate, chlorotoluron, dimefuron, diuron, linuron, fluometuron, isoproturon, isouron, tebuthiuron, benzthiazuron, methabenzthiazuron, propanil, metobromuron, metoxuron, monolinuron, siduron, simazine, atrazine, propazine, cyanazine, ametryn, simetryn, dimethametryn, prometryn, terbumeton, terbuthylazine, terbutryn, trietazine, hexazinone, metamitron, metribuzin, amicarbazone, bromacil, lenacil, terbacil, chloridazon, desmedipham, phenmedipham, and agriculturally acceptable salts and derivatives thereof.

In another embodiment, the B-7 group includes a herbicide from Group B selected from propachlor, metazachlor, alachlor, acetochlor, metolachlor, S-metolachlor, butachlor, pretilachlor, thenylchlor, indanofan, cafenstrole, fentrazamide, dimethenamid, dimethenamid-P, mefenacet, pyroxasulfone, fenoxasulfone, naproanilide, napropamide, anilofos, flufenacet, ipfencarbazone, and agriculturally acceptable salts and derivatives thereof.

In another embodiment, the B-8 group includes a herbicide from Group B selected from trifluralin, pendimethalin, ethalfluralin, benfluralin, oryzalin, prodiamine, butamifos, dithiopyr, thiazopyr, and agriculturally acceptable salts and derivatives thereof.

In another embodiment, the B-9 group includes a herbicide from Group B selected from 2,4-dichlorophenoxyacetic acid (2,4-D) and its salts and esters, 4-(2,4-dichlorophenoxy)butyric acid (2,4-DB) and its salts or esters (dimethylammonium salt, isooctyl ester, and choline salt), 3,6-dichloro-2-methoxybenzoic acid (Dicamba) and its salts (dicholamine or N,N-bis-(3-aninopropyl)methylamine salts), 2-methyl-4-chlorophenoxyacetic acid (MCPA) and its salts or esters (dimethylammonium salt, 2-ethylhexyl ester, isooctyl ester, sodium salt, and choline salt), 4-(4-chloro-o-tolyloxy)butyric acid (MCPB), mecoprop and its salts or esters (dimethylammonium salt, dioramine salt, ethadyl ester, 2-ethylhexyl ester, isooctyl ester, methyl ester, potassium salt, sodium salt, trolamine salt, and choline salt), mecoprop-P and its salts or esters (dimethylammonium salt, 2-ethylhexyl ester, isobutyl salt, potassium salt, and choline salt), dichlorprop and its salt or ester (butotyl ester, dimethylammonium salt, 2-ethylhexyl ester, isooctyl ester, methyl ester, potassium salt, sodium salt, and choline salt), dichlorprop-P, dichlorprop-P dimethylammonium, triclopyr and its salts or esters (butotyl ester, and triethylammonium salt), fluroxypyr, fluroxypyr-meptyl, picloram and its salts (potassium salt, tris(2-hydroxypropyl)ammonium salt, and choline salt), quinclorac, quinmerac, aminopyralid and its salts (potassium salt, tris(2-hydroxypropyl)ammonium salt, and choline salt), clopyralid and its salts (olamine salt, potassium salt, triethylammonium salt, and choline salt), clomeprop, aminocyclopyrachlor, halauxifen, halauxifen-methyl, florpyrauxifen, and florpyrauxifenben, and agriculturally acceptable salts and derivatives thereof.

In another embodiment, the B-10 group includes a herbicide from Group B selected from glyphosate, glyphosate-isopropylammonium salt, glyphosate-trimesium salt, glyphosate-ammonium salt, glyphosate-diammonium salt, glyphosate-dimethylammonium salt, glyphosate-monoethanolamine salt, glyphosate-sodium salt, glyphosate-potassium salt, glyphosate-guanidine salt, and other agriculturally acceptable salts and derivatives thereof.

In another embodiment, the B-11 group includes a herbicide from Group B selected from glufosinate, glufosinate-ammonium salt, glufosinate-P, glufosinate-P-sodium salt, and bialaphos, and agriculturally acceptable salts and derivatives thereof.

In another embodiment, the B-12 group includes a herbicide from Group B selected from isoxaben, dichlobenil, methiozolin, diallate, butylate, triallate, chlorpropham, asulam, phenisopham, benthiocarb, molinate, esprocarb, pyributicarb, prosulfocarb, orbencarb, ethylsulfanyl-N,N-dipropylformamide (EPTC), dimepiperate, swep, difenoxuron, methyldymron, bromobutide, daimuron, cumyluron, diflufenzopyr, diflufenzopyr-sodium salt, etobenzanid, tridiphane, amitrole, clomazone, broclozone, 2-[(2,4-dichlorophenyl) methyl]-4,4-dimethylisoxazolidin-3-one (CAS Registry Number: 81777-95-9), (3S,4S)—N-(2-fluorophenyl)-1-methyl-2-oxo-4-[3-(trifluoromethyl)phenyl]-3-pyrrolidinecarboxamide (CAS Registry Number: 2053901-33-8), maleic hydrazide, oxaziclomefone, cinmethylin, benfuresate, dalapon, chlorthiamid, flupoxam, bensulide, paraquat, paraquat-dichloride, diquat, diquat-dibromide, monosodium methanearsonate (MSMA), indaziflam, and triaziflam, and agriculturally acceptable salts and derivatives thereof.

In another embodiment, the herbicidal composition includes a safener from Group C selected from benoxacor, cloquintocet, cyometrinil, cyprosulfamide, dichlormid, dicyclonone, dietholate, fenchlorazole, fenclorim, flurazole, fluxofenim, furilazole, isoxadifen, mefenpyr, mephenate, naphthalic anhydride, oxabetrinil, 4-(dichloroacetyl)-1-oxa-4-azaspiro[4.5]decane, 2,2,5-trimethyl-3-(dichloroacetyl)-1,3-oxazolidine, and N-(2-methoxybenzoyl)-4-[(methylaminocarbonyl)amino]benzenesulfon-amide.

In another aspect, provided is a method for controlling weeds that includes the step of applying a composition including the compound of formula (I), a herbicide compound from group B, and optionally a safener from group C to a place where weeds are growing or to grow in a crop field, a vegetable field, a land under perennial crops, a non-crop land, or the like. In a crop field and a vegetable field, the present composition may be applied before, simultaneously with, and/or after sowing a crop seed.

In one embodiment, the compositions of the invention are directed to a method for controlling a ‘volunteer’ crop. Such ‘volunteer’ crops are defined as undesirable seedlings and plants originating from seeds, tubers, roots, or other plant portions and organs, remaining in the field from previous farming and cropping practices. For example, in one embodiment the invention is directed to controlling volunteer corn (Zea mays) in an area where corn growth is undesirable. An example of undesirable volunteer corn growth would be in a field where soybeans are growing. Other examples include methods of controlling volunteer crops such as Sorghum (Sorghum bicolor), Wheat (Triticum aestivum), Durum Wheat (Triticum turgidum spp. durum), Rye (Secale cereale), Triticale (x Triticosecale Wittmack), Barley (Hordeum vulgare), Oats (Avena sativa), Millet (Setaria italica), Carinata (Brassica carinata), Camelina (Camelina sativa), Canola (Brassica napus), Flax (Linum usitatissimum), Soybean (Glycine max), Cotton (Gossypium hirsutum), Industrial Hemp (Cannabis sativa), Carinata (Brassica carinata), Sugarcane (Saccharum officinarum), Potato (Solanum tuberosum), Rice (Oryza sativa), Alfalfa (Medicago sativa), Pennycress (Thlaspi arvense), Clover (Trifolium repens), Sunflower (Heliantus annus), Peanut (Arachis hypogaea)

In another embodiment, the composition is a formulation prepared by mixing the compound of formula (I) and the at least one compound selected from herbicide compound group B, and optionally safener group C with a carrier such as a solid carrier or a liquid carrier, and adding an auxiliary agent for formulation such as a surfactant if necessary. Preferable formulation types of such a formulation are aqueous liquid suspension concentrates, wettable powders, water dispersible granules, granules, and emulsifiable concentrates. The present composition may be used in combination with a formulation containing another herbicide as an active ingredient.

In another embodiment, the total content of the compound of formula (I) and the at least one compound selected from the group consisting of the herbicide compound group B and the safener group C in the present composition is usually within a range of 0.01 to 90% by weight. In another embodiment, the total content of the compound of formula (I) is 1 to 80% by weight. When the at least one compound is selected from the group of herbicide group B is a salt (for example, glyphosate-potassium salt), the weight of the at least one compound is represented by the acid equivalent.

In some embodiments, the mixing ratio of a compound of formula (I) to the at least one compound selected from herbicide compound group B include about 1:0.1, about 1:0.2, about 1:0.3, about 1:0.5, about 1:0.6, about 1:0.7, about 1:0.8, about 1:1, about 1:1.2, about 1:1.4, about 1:1.6, about 1:1.8, about 1:2, about 1:2.2, about 1:2.4, about 1:2.6, about 1:2.8, about 1:3, about 1:5, about 1:7, about 1:10, about 1:15, about 1:20, about 1:30, and about 1:50 by weight ratio. The word “about” means that the specified ratio includes the ratio in the range increased or decreased by 10% by weight relative to the specified ratio. For example, a ratio of about 1:2 includes a range of 1:1.8 to 1:2.2.

In certain embodiments, the combination of the compound of formula (I) and the herbicide selected from herbicide group B include: a combination of the compound of formula (I) and pyrithiobac (1:0.1 to 1:20); a combination of the compound of formula (I) and pyrithiobac-sodium salt (1:0.1 to 1:20); a combination of the compound of formula (I) and chlorimuron-ethyl (1:0.1 to 1:20); a combination of the compound of formula (I) and foramsulfuron (1:0.1 to 1:20); a combination of the compound of formula (I) and halosulfuron-methyl (1:0.1 to 1:20); a combination of the compound of formula (I) and nicosulfuron (1:0.1 to 1:20); a combination of the compound of formula (I) and primisulfuron-methyl (1:0.1 to 1:20); a combination of the compound of formula (I) and rimsulfuron (1:0.1 to 1:20); a combination of the compound of formula (I) and trifloxysulfuron-sodium salt (1:0.1 to 1:20); a combination of the compound of formula (I) and trifludimoxazin (1:0.1 to 1:20); a combination of the compound of formula (I) and chlorsulfuron (1:0.1 to 1:20); a combination of the compound of formula (I) and iodosulfuron-methyl-sodium (1:0.1 to 1:20); a combination of the compound of formula (I) and iofensulfuron-sodium (1:0.1 to 1:20); a combination of the compound of formula (I) and metsulfuron-methyl (1:0.1 to 1:20); a combination of the compound of formula (I) and prosulfuron (1:0.1 to 1:20); a combination of the compound of formula (I) and thifensulfuron-methyl (1:0.1 to 1:20); a combination of the compound of formula (I) and tribenuron-methyl (1:0.1 to 1:20); a combination of the compound of formula (I) and thiencarbazone-methyl (1:0.1 to 1:20); a combination of the compound of formula (I) and cloransulam-methyl (1:0.1 to 1:20); a combination of the compound of formula (I) and flumetsulam (1:0.1 to 1:20); a combination of the compound of formula (I) and imazamethabenz-methyl (1:0.1 to 1:20); a combination of the compound of formula (I) and imazamox-ammonium salt (1:0.1 to 1:20); a combination of the compound of formula (I) and imazapic-ammonium salt (1:0.1 to 1:20); a combination of the compound of formula (I) and imazapyr-isopropylammonium salt (1:0.1 to 1:20); a combination of the compound of formula (I) and imazaquin-ammonium salt (1:0.1 to 1:20); a combination of the compound of formula (I) and imazethapyr-ammonium salt (1:0.1 to 1:20); a combination of the compound of formula (I) and fenoxaprop-ethyl (1:0.1 to 1:20); a combination of the compound of formula (I) and fenoxaprop-P-ethyl (1:0.1 to 1:20); a combination of the compound of formula (I) and fluazifop-butyl (1:0.1 to 1:20); a combination of the compound of formula (I) and fluazifop-P-butyl (1:0.1 to 1:20); a combination of the compound of formula (I) and quizalofop-ethyl (1:0.1 to 1:20); a combination of the compound of formula (I) and quizalofop-P-ethyl (1:0.1 to 1:20); a combination of the compound of formula (I) and clethodim (1:0.1 to 1:20); a combination of the compound of formula (I) and sethoxydim (1:0.1 to 1:20); a combination of the compound of formula (I) and carfentrazone-ethyl (1:0.1 to 1:20); a combination of the compound of formula (I) and saflufenacil (1:0.1 to 1:20); a combination of the compound of formula (I) and sulfentrazone (1:0.1 to 1:20); a combination of the compound of formula (I) and pyraflufen-ethyl (1:0.1 to 1:20); a combination of the compound of formula (I) and fluthiacet-methyl (1:0.1 to 1:20); a combination of the compound of formula (I) and flufenpyr-ethyl (1:0.1 to 1:20); a combination of the compound of formula (I) and flumiclorac-pentyl (1:0.1 to 1:20); a combination of the compound of formula (I) and flumioxazin (1:0.1 to 1:20); a combination of the compound of formula (I) and oxyfluorfen (1:0.1 to 1:30); a combination of the compound of formula (I) and acifluorfen-sodium salt (1:0.1 to 1:30); a combination of the compound of formula (I) and fomesafen-sodium salt (1:0.1 to 1:30); a combination of the compound of formula (I) and lactofen (1:0.1 to 1:30); a combination of the compound of formula (I) and tiafenacil (1:0.1 to 1:20); a combination of the compound of formula (I) and epyrifenacil (1:0.1 to 1:20); a combination of the compound of formula (I) and bicyclopyrone (1:0.1 to 1:20); a combination of the compound of formula (I) and tembotrione (1:0.1 to 1:20); a combination of the compound of formula (I) and mesotrione (1:0.1 to 1:20); a combination of the compound of formula (I) and isoxaflutole (1:0.1 to 1:20); a combination of the compound of formula (I) and topramezone (1:0.1 to 1:20); a combination of the compound of formula (I) and tolpyralate (1:0.1 to 1:20); a combination of the compound of formula (I) and fenquinotrione (1:0.1 to 1:20); a combination of the compound of formula (I) and lancotrione-sodium salt (1:0.1 to 1:20); a combination of the compound of formula (I) and 2-methyl-N-(5-methyl-1,3,4-oxadiazol-2-yl)-3-(methylsulfonyl)-4-(trifluoromethyl)benzamide (1:0.1 to 1:20); a combination of the compound of formula (I) and 2-chloro-N-(1-methyl-1H-tetrazol-5-yl)-3-(methylthio)-4-(trifluoromethyl)benzamide (1:0.1 to 1:20); a combination of the compound of formula (I) and 4-(4-fluorophenyl)-6-[(2-hydroxy-6-oxo-1-cyclohexene-1-yl)carbonyl]-2-methyl-1,2,4-triazine-3,5(2H,4H)-dione (1:0.1 to 1:20); a combination of the compound of formula (I) and norflurazon (1:0.1 to 1:20); a combination of the compound of formula (I) and fluridone (1:0.1 to 1:20); a combination of the compound of formula (I) and bentazone (1:1 to 1:50); a combination of the compound of formula (I) and bromoxynil octanoate (1:1 to 1:50); a combination of the compound of formula (I) and diuron (1:1 to 1:50); a combination of the compound of formula (I) and linuron (1:1 to 1:50); a combination of the compound of formula (I) and fluometuron (1:1 to 1:50); a combination of the compound of formula (I) and simazine (1:1 to 1:50); a combination of the compound of formula (I) and atrazine (1:1 to 1:50); a combination of the compound of formula (I) and ametryn (1:1 to 1:50); a combination of the compound of formula (I) and prometryn (1:1 to 1:50); a combination of the compound of formula (I) and metribuzin (1:1 to 1:50); a combination of the compound of formula (I) and alachlor (1:1 to 1:50); a combination of the compound of formula (I) and acetochlor (1:1 to 1:50); a combination of the compound of formula (I) and metolachlor (1:1 to 1:50); a combination of the compound of formula (I) and S-metolachlor (1:1 to 1:50); a combination of the compound of formula (I) and dimethenamid (1:1 to 1:50); a combination of the compound of formula (I) and dimethenamid-P (1:1 to 1:50); a combination of the compound of formula (I) and pyroxasulfone (1:0.1 to 1:20); a combination of the compound of formula (I) and flufenacet (1:0.1 to 1:20); a combination of the compound of formula (I) and trifluralin (1:1 to 1:50); a combination of the compound of formula (I) and pendimethalin (1:1 to 1:50); a combination of the compound of formula (I) and ethalfluralin (1:1 to 1:50); a combination of the compound of formula (I) and 2,4-DB (1:1 to 1:50); a combination of the compound of formula (I) and fluroxypyr (1:1 to 1:50); a combination of the compound of formula (I) and fluroxypyrmeptyl (1:1 to 1:50); a combination of the compound of formula (I) and clopyralid-potassium salt (1:1 to 1:50); a combination of the compound of formula (I) and clopyralid-olamine salt (1:1 to 1:50); a combination of the compound of formula (I) and clopyralid-triethylammonium salt (1:1 to 1:50); a combination of the compound of formula (I) and halauxifen (1:0.1 to 1:20); a combination of the compound of formula (I) and halauxifen-methyl (1:0.1 to 1:20); a combination of the compound of formula (I) and florpyrauxifen (1:0.1 to 1:20); a combination of the compound of formula (I) and florpyrauxifen-benzyl (1:0.1 to 1:20); a combination of the compound of formula (I) and glyphosate (1:1 to 1:50); a combination of the compound of formula (I) and glyphosate-isopropylammonium salt (1:1 to 1:50); a combination of the compound of formula (I) and glyphosate-ammonium salt (1:1 to 1:50); a combination of the compound of formula (I) and glyphosate-dimethylamine salt (1:1 to 1:50); a combination of the compound of formula (I) and glyphosate-monoethanolamine salt (1:1 to 1:50); a combination of the compound of formula (I) and glyphosate-potassium salt (1:1 to 1:50); a combination of the compound of formula (I) and glyphosate-guanidine salt (1:1 to 1:50); a combination of the compound of formula (I) and glufosinate (1:1 to 1:50); a combination of the compound of formula (I) and glufosinate-P (1:1 to 1:50); a combination of the compound of formula (I) and glufosinate-P-sodium salt (1:1 to 1:50); a combination of the compound of formula (I) and EPTC (1:1 to 1:50); a combination of the compound of formula (I) and diflufenzopyr (1:1 to 1:50); a combination of the compound of formula (I) and diflufenzopyr-sodium salt (1:1 to 1:50); a combination of the compound of formula (I) and clomazone (1:1 to 1:50); a combination of the compound of formula (I) and 2-[(2,4-dichlorophenyl)methyl]-4,4-dimethylisoxazolidin-3-one (1:1 to 1:50); a combination of the compound of formula (I) and (3S,4S)—N-(2-fluorophenyl)-1-methyl-2-oxo-4-[3-(trifluoromethyl)phenyl]-3-pyrrolidinecarboxamide (1:1 to 1:50); a combination of the compound of formula (I) and cinmethylin (1:1 to 1:50); a combination of the compound of formula (I) and MSMA (1:1 to 1:50); a combination of the compound of formula (I) and paraquat (1:1 to 1:50); a combination of the compound of formula (I) and paraquat-dichloride (1:1 to 1:50); a combination of the compound of formula (I) and diquat (1:1 to 1:50); a combination of the compound of formula (I) and diquat-dibromide (1:0.1 to 1:20); a combination of the compound of formula (I) and benoxacor (1:0.1 to 1:20); a combination of the compound of formula (I) and cyprosulfamide (1:0.1 to 1:20); and a combination of the compound of formula (I) and isoxadifen-ethyl (1:0.1 to 1:20).

In one embodiment, the herbicide selected from herbicide group B is glyphosate and the weight ratio of Compound 1 to glyphosate is about 30:60 to 1000:1120.

In another embodiment, the herbicide selected from herbicide group B is pyroxasulfone and the weight ratio of Compound 1 to pyroxasulfone is about 15:60 to 90.

In another embodiment, the herbicide selected from herbicide group B is mesotrione and the weight ratio of Compound 1 to mesotrione is about 30:60 to 105:210.

In another embodiment, the herbicide selected from herbicide group B is glufosinate and the weight ratio of Compound 1 to glufosinate is about 30:60 to 450.

In another embodiment, the herbicide selected from herbicide group B is dicamba and the weight ratio of Compound 1 to dicamba is about 30:60 to 280.

In another embodiment, the herbicide selected from herbicide group B is 2,4-D acid and the weight ratio of Compound 1 to 2,4-D acid is about 30:60 to 280.

In another embodiment, the herbicide selected from herbicide group B is 2,4-D ester and the weight ratio of Compound 1 to 2,4-D ester is about 30:60 to 280.

In another embodiment, the herbicide selected from herbicide group B is S-metolachlor and the weight ratio of Compound 1 to S-metolachlor is about 30 to 1000.

In yet another embodiment, the herbicide selected from herbicide group B is metribuzin and the weight ratio of Compound 1 to metribuzin is about 30 to 210.

In one embodiment, the undesired vegetation treated by the compositions of the invention comprises protoporphyrinogen IX oxidase (PPO) inhibitor-resistant weeds. In a further embodiment, the PPO inhibitor-resistant weeds have a dG210 mutation.

Before, simultaneously with, and/or after sowing a crop seed treated with one or more compounds selected from the group consisting of an insecticide compound, a nematicide compound, a fungicide compound, and the like, a composition including a compound of formula (I) may be applied to the field in which the crop seed have been sown or is to be sown. Such compounds include neonicotinoid compounds, diamide compounds, carbamate compounds, oiganophosphorus compounds, biological nematicide compounds, azole compounds, strobilurin compounds, metalaxyl compounds, and SDHI compounds.

Examples of crop fields treated by compositions that include a compound of formula (I) include edible crop fields such as peanut fields, soybean fields, corn fields, and wheat fields, feed crop fields such as sorghum fields and oat fields, industrial crop fields such as cotton fields and rape fields, and sugar crop fields such as sugarcane fields and sugar beet fields.

Examples of the vegetable fields treated by compositions that include fields for cultivation of solanaceous vegetables (eggplants, tomatoes, bell peppers, capsicums, potatoes, and the like), fields for cultivation of cucurbitaceous vegetables (cucumbers, pumpkins, zucchini, watermelons, melons, and the like), fields for cultivation of cruciferous vegetables (radishes, turnips, horseradishes, kohlrabies, Chinese cabbages, cabbages, mustard, broccolis, cauliflowers, and the like), fields for cultivation of asteraceous vegetables (burdocks, garland chrysanthemums, artichokes, lettuces, and the like), fields for cultivation of liliaceous vegetables (leeks, onions, garlics, and asparagus), fields for cultivation of apiaceous vegetables (carrots, parsley, celery, parsnips, and the like), fields for cultivation of chenopodiaceous vegetables (spinach, chards, and the like), fields for cultivation of lamiaceous vegetables (perilla, mint, basil, and lavender), strawberry fields, sweet potato fields, yam fields, and taro fields.

Examples of the land under perennial crops treated by compositions that include a compound of formula (I) include orchards, tea fields, mulberry fields, coffee fields, banana fields, palm fields, flowering tree firms, flowering tree fields, planting stock fields, nursery fields, forest lands, and gardens. Examples of the orchard trees include pomaceous fruits (apples, pears, Japanese pears, Chinese quinces, quinces, and the like), stone fruits (peaches, plums, nectarines, Japanese apricots, cherries, apricots, prunes, and the like), citrus fruits (Citrus unshiu, oranges, lemons, limes, grapefruits, and the like), nut trees (chestnuts, walnuts, hazelnut trees, almonds, pistachios, cashew nut trees, macadamia nut trees, and the like), berry fruits (grapes, blueberries, cranberries, blackberries, raspberries, and the like), Japanese persimmons, olives, and loquats.

Examples of the non-crop land treated by compositions that include a compound of formula (I) include athletic fields, empty lots, railroad edges, parks, parking lots, road edges, dry riverbeds, lands under a power line, residential lands, and factory sites.

The crop cultivated in the crop field treated by compositions comprising a compound of formula (I) is not limited as long as the crop is a variety generally cultivated as a crop. It may be a plant that can be produced by natural crossing, a plant that can be generated by mutation, an Fl hybrid plant, or a transgenic plant (also referred to as a genetically-modified plant). The plant generally has properties such as obtaining of the tolerance to a herbicide, accumulation of a toxic substance against a pest, suppression of the susceptibility to a disease, increase in the yield potential, improvement in the tolerance to a biotic and an abiotic stressors, accumulation of a substance, and improvement in the preservability and the processability.

Examples of the plants to which herbicide tolerance has been imparted by genetic engineering technique include plants to which tolerance has been imparted to 4-hydroxyphenylpyruvate dioxygenase (HPPD) inhibitors such as isoxaflutole and mesotrione, acetolactate synthase (ALS) inhibitors such as imidazolinone herbicides containing imazethapyr and sulfonylurea herbicides containing thifensulffiron-methyl, 5-enolpyruvylshikimate-3-phosphate synthase (EPSP) inhibitors such as glyphosate, glutamine synthase inhibitors such as glufosinate, auxin herbicides such as 2,4-D and dicamba, and oxynyl herbicides containing bromoxynil. Herbicide-tolerant transgenic plants include cereals such as wheat, barley, rye, and oats, canola, sorghum, soybeans, rice, rape, sugar beet, sugar cane, grapes, lentils, sunflowers, alfalfa, pomaceous fruits, drupes, coffee, tea, strawberries, lawn grass, tomatoes, potatoes, cucumbers, and vegetables such as lettuces, and more preferable herbicide-tolerant transgenic plants are cereals such as wheat, barley, rye, and oats, soybeans, rice, vines, tomatoes, potatoes, and pomaceous fruits.

Specific herbicide-tolerant plants treated by compositions comprising a compound of formula (I) include Glyphosate herbicide-tolerant plants. In order to obtain the glyphosate herbicide-tolerant plants, one or more genes are introduced among a glyphosate-tolerant EPSPS gene (CP4 epsps) from Agrobacterium tumefaciens strain CP4, a glyphosate metabolizing enzyme gene (gat4601, gat4621) in which the metabolic activity of the glyphosate metabolizing enzyme (glyphosate N-acetyltransferase) gene from Bacillus Uchenformis is enhanced by a shuffling technique, a glyphosate metabolizing enzyme (glyphosate oxidase gene, goxv247) from Ochrobacterum anthropi strain LBAA, and EPSPS genes that are from maize and have glyphosate-tolerant mutation (mepsps, 2mepsps). Main examples of such modified plants include alfalfa (Medicago sativa), Argentine canola (Brassica napus), cotton (Gossypium hirsutum L.), creeping bentgrass (Agrostis stolonfera), maize (Zea mays L.), polish canola (Brassica rapa), a potato (Solanum tuberosum L.), a soybean (Glycine max L.), sugar beet (Beta vulgaris), and wheat (Triticum aestivum). Some glyphosate-tolerant transgenic plants are commercially available. For example, the genetically-modified plant in which the glyphosate-tolerant EPSPS from the Agrobacterium is expressed is commercially available with a trade name such as “Roundup Ready®”, the genetically-modified plant in which the glyphosate metabolizing enzyme that is from the Bacillus and has the metabolic activity enhanced by a shuffling technique is expressed is commercially available with a trade name such as “Optimum® GAT™” or “Optimum® Gly canola”, and the genetically-modified plant in which the EPSPS that is from maize and has glyphosate-tolerant mutation is expressed is commercially available with the trade name “GlyTol™”.

Other herbicide-tolerant plants treated by compositions comprising a compound of formula (I) include Glufosinate herbicide-tolerant plants. In order to obtain the glufosinate herbicide-tolerant plants, one or more genes are introduced among a phosphinothricin N-acetyltransferase (PAT) gene (bar) that is a glufosinate metabolizing enzyme from Streptomyces hygroscopicus, a phosphinothricin N-acetyltransferase (PAT) enzyme gene (pat) that is a glufosinate metabolizing enzyme from Streptomyces viridochromogenes, and a synthesized pat gene (pat syn) from Streptomyces viridochromogenes strain Tu494. Main examples of such plants are Argentine canola (Brassica napus), chicory (Cichorium intybus), cotton (Gossypium hirsutum L.), maize (Zea mays L.), polish canola (Brassica rapa), rice (Oryza sativa L.), soybean (Glycine max L.), and sugar beet (Beta vulgaris). Some glufosinate-tolerant genetically-modified plants are commercially available. The genetically-modified plant from a glufosinate metabolizing enzyme (bar) from Streptomyces hygroscopicus, and from Streptomyces viridochromogenes is commercially available with a trade name such as “LibertyLink™”, “InVigor™”, or “WideStrike™”.

Herbicide-tolerant plants treated by compositions comprising a compound of formula (I) also include Oxynil herbicide-tolerant (e.g., bromoxynil-tolerant) plants. Bromoxynil-tolerant transgenic plants into which a nitrilase gene (bxn) is introduced include those having an oxynil herbicide metabolizing enzyme from Klebsiellapneumoniae subsp. ozaenae. Such examples include Argentine canola (Brassica napus), cotton (Gossypium hirsutum L.), and tobacco (Nicotiana tabacum L.). Such plants are commercially available with trade names like “Navigator™ canola” or “BXN™”.

Herbicide-tolerant plants treated by compositions comprising a compound of formula (I) also include ALS herbicide-tolerant plants such as carnations (Dianthus caryophyllus) into which an ALS herbicide-tolerant ALS gene (surB) as a selection marker from tobacco (Nicotiana tabacum) is introduced and are commercially available with the trade names “Moondust™”, “Moonshadow™”, “Moonshade™”, “Moonlite™” “Moonaqua™”, “Moonvista™”, “Moonique™”, “Moonpearl™”, “Moonberry™”, and “Moonvelvet™”; flax (Linum usitatissumum L.) into which an ALS herbicide-tolerant ALS gene (als) from Arabidopsis thaliana is introduced and is commercially available with the trade name “CDC Triffid Flax”; sulfonylurea herbicide-tolerant and an imidazolinone herbicide-tolerant maize (Zea mays L.) into which an ALS herbicide-tolerant ALS gene (zm-hra) from maize is introduced and is commercially available with the trade name “Optimum™ GAT™”; an imidazolinone herbicide-tolerant soybean into which an ALS herbicide-tolerant ALS gene (csrl-2) from Arabidopsis thaliana is introduced and is commercially available with the trade name “Cultivance™”; sulfonylurea herbicide-tolerant soybeans into which an ALS herbicide-tolerant ALS gene (gm-hra) from a soybean (Glycine max) is introduced are commercially available with the trade names “Treus™”, “Plenish™” and “Optimum GAT™”; and cotton into which an ALS herbicide-tolerant ALS gene (S4-FlrA) from tobacco (Nicotiana tabacum cv. Xanthi) is introduced.

Herbicide-tolerant plants treated by compositions comprising a compound of formula (I) also include HPPD herbicide-tolerant plants such as a soybean into which a mesotrione-tolerant HPPD gene (avhppd03) from an oat (Avena sativa) and a phinothricin N-acetyltransferase (PAT) enzyme gene (pat) are simultaneously introduced and soybean tolerant to mesotrione that has a glufosinate metabolizing enzyme from Streptomyces viridochromogenes and is commercially available with the trade name “Herbicide-tolerant Soybean line.”

Herbicide-tolerant plants treated by compositions comprising a compound of formula (I) also include 2,4-dichlorophenoxyacetic acid (2,4-D)-tolerant plants such as maize into which an aryloxyalkanoate dioxygenase gene (aad-1) that is a 2,4-D metabolizing enzyme from Sphingobium herbicidovorans is introduced and is commercially available with the trade name “Enlist™ Maize” and a soybean and cotton into which an aryloxyalkanoate dioxygenase gene (aad-12) that is a 2,4-D metabolizing enzyme from Delftia acidovorans is introduced and is commercially available with the trade name “Enlist™ Soybean.”

Herbicide-tolerant plants treated by compositions comprising a compound of formula (I) also include Dicamba-tolerant plants such as a soybean and cotton into which a dicamba monooxygenase gene (dmo) that is a dicamba metabolizing enzyme from Stenotrophomonas maltophilia strain DI-6 is introduced; a soybean (Glycine max L.) into which a glyphosate-tolerant EPSPS gene (CP4 epsps) from Agrobacterium tumefaciens strain CP4 is introduced simultaneously with the above-mentioned gene and is commercially available with the trade name “Genuity™ Roundup Ready™ 2 Xtend™”.

Examples of the commercially available transgenic plants to which herbicide tolerance has been imparted and can be treated by compositions comprising a compound of formula (I) include the glyphosate-tolerant maize “Roundup Ready Com”, “Roundup Ready 2”, “Agrisure GT”, “Agrisure GT/CB/LL”, “Agrisure GT/RW”, “Agrisure 3000GT”, “YieldGard VT Rootworm/RR2”, and “YieldGard VT Triple”; the glyphosate-tolerant soybeans “Roundup Ready Soybean” and “Optimum GAT”; the glyphosate-tolerant cotton “Roundup Ready Cotton” and “Roundup Ready Flex”; the glyphosate-tolerant canola “Roundup Ready Canola”; the glyphosate-tolerant alfalfa “Roundup Ready Alfalfa”, the glyphosate-tolerant rice “Roundup Ready Rice”; the glufosinate-tolerant maize “Roundup Ready 2”, “Liberty Link”, “Eierculex 1”, “Eierculex RW”, “Eierculex Xtra”, “Agrisure GT/CB/LL”, “Agrisure CB/LL/RW”, and “Bt10”; the glufosinate-tolerant cotton “FiberMax Liberty Link”; the glufosinate-tolerant rice “Liberty Link Rice”; the glufosinate-tolerant canola “in Vigor”; the glufosinate-tolerant rice “Liberty Link Rice”; the bromoxynil-tolerant cotton “BXN”; the bromoxynil-tolerant canola “Navigator” and “Compass”; and the glufosinate-tolerant canola “InVigor.”

Additional plants modified with respect to a herbicide are widely known and can be treated by compositions comprising a compound of formula (I), the examples of which include alfalfa, apples, barley, eucalyptuses, flax, grapes, lentils, rape, peas, potatoes, rice, sugar beet, sunflowers, tobacco, tomato, turfgrass, and wheat that are tolerant to glyphosate (see, for example, U.S. Pat. Nos. 5,188,642, 4,940,835, 5,633,435, 5,804,425, and 5,627,061); beans, cotton, soybeans, peas, potatoes, sunflowers, tomatoes, tobacco, maize, sorghum, and sugar cane that are tolerant to dicamba (see, for example, WO2008051633, U.S. Pat. Nos. 7,105,724, and 5,670,454); soybeans, sugar beet, potatoes, tomatoes, and tobacco that are tolerant to glufosinate (see, for example, U.S. Pat. Nos. 6,376,754, 5,646,024, and 5,561,236); cotton, peppers, apples, tomatoes, sunflowers, tobacco, potatoes, maize, cucumbers, wheat, soybeans, sorghum, and cereals that are tolerant to 2,4-D (see, for example, U.S. Pat. Nos. 6,153,401, 6,100,446, WO2005107437, U.S. Pat. Nos. 5,608,147, and 5,670,454); and canola, maize, millet, barley, cotton, mustard, lettuces, lentils, melons, millet, oats, sword beans, potatoes, rice, rye, sorghum, soybeans, sugar beet, sunflowers, tobacco, tomatoes, and wheat that are tolerant to acetolactate synthase (ALS) inhibitor herbicide (for example, a sulfonylurea herbicide and an imidazolinone herbicide) (see, for example, U.S. Pat. No. 5,013,659, WO2006060634, U.S. Pat. Nos. 4,761,373, 5,304,732, 6,211,438, 6,211,439, and 6,222,100); rice tolerant to an imidazolinone herbicide, the examples including rice having specific mutation (for example, S653N, S654K, A122T, S653(At)N, S654(At)K, and A122(At)T) in the acetolactate synthase gene (acetohydroxyacid synthase gene) (see, for example, US 2003/0217381, and W0200520673); and barley, sugar cane, rice, maize, tobacco, soybeans, cotton, rape, sugar beet, wheat, and potatoes that are tolerant to an HPPD inhibitor herbicide (for example, an isoxazole herbicide such as isoxaflutole, a triketone herbicide such as sulcotrione or mesotrione, a pyrazole herbicide such as pyrazolynate, or diketonitrile that is a decomposition product of isoxaflutole) (see, for example, WO2004/055191, W0199638567, WO1997049816, and U.S. Pat. No. 6,791,014).

Examples of the plants to which herbicide tolerance has been imparted by a classical technique or a genome breeding technique that can be treated by compositions comprising a compound of formula (I) include the rice “Clearfield® Rice”, the wheat “Clearfield® Wheat”, the sunflower “Clearfield® Sunflower”, the lentil “Clearfield® lentils”, and the canola “Clearfield® canola” (manufactured by BASF SE) that are tolerant to an imidazolinone-based ALS inhibitor herbicide such as imazethapyr or imazamox; the soybean “STS® soybean” that is tolerant to a sulfonyl-based ALS inhibitor herbicide such as thifensulfuron-methyl; the sethoxydim-tolerant maize “SR® corn” and ‘Poast Protected® corn” that are tolerant to an acetyl-CoA carboxylase inhibitor such as a trionoxime herbicide or an aryloxy phenoxypropionic acid herbicide; the sunflower “ExpressSun®” that is tolerant to a sulfonylurea herbicide such as tribenuron; the rice “Provisia™ Rice” that is tolerant to an acetyl-CoA carboxylase inhibitor such as quizalofop; and the canola “Triazine Tolerant Canola” that is tolerant to a PSII inhibitor.

Examples of the plants to which herbicide tolerance has been imparted by a genome editing technique that can be treated by compositions comprising a compound of formula (I) include include the canola “SU Canola®” tolerant to a sulfonylurea herbicide in which a rapid variety development technique (Rapid Trait Development System, RTDS®) is used. RTDS® corresponds to oligonucleotide-directed mutagenesis of the genome editing technique, and by RTDS, it is possible to introduce mutation in a DNA in a plant via Gene Repair Oligonucleotide (GRON), that is, a chimeric oligonucleotide of the DNA and the RNA without cutting the DNA. In addition, examples of the plants include maize in which herbicide tolerance and phytic acid content have been reduced by deleting the endogenous gene IPK1 using zinc finger nuclease (see, for example, Nature 459, 437-441 2009); and rice to which herbicide tolerance has been imparted using CRISPR-Cas9.

Examples of a crop tolerant to a specific PPO inhibitor that can be treated by compositions comprising a compound of formula (I) include crops to which PPO having a reduced affinity for the inhibitor is imparted by a genetic engineering technique. Alternatively, the crop may have a substance that detoxifies and decomposes the PPO inhibitor by cytochrome P450 monooxygenase alone or in combination with the above-mentioned PPO. The tolerant crops are described in, for example, patent documents such as WO2011085221, WO2012080975, WO2014030090, WO2015022640, WO2015022636, WO2015022639, WO2015092706, WO2016203377, WO2017198859, WO2018019860, WO2018022777, WO2017112589, WO2017087672, WO2017039969, and WO2017023778, and non-patent document Li & Nicholl in Pest Management Science (2005), Vol. 61, pgs. 277-285.

Examples of plants that can be treated by compositions comprising a compound of formula (I) include those in which herbicide tolerance has been imparted by a new breeding technique in which the property of a GM rootstock is imparted to a scion by a breeding technique in which grafting is used include the non-transgenic soybean scion to which glyphosate tolerance is imparted using the glyphosate-tolerant soybean Roundup Ready® as a rootstock (see Jiang, et al., in Weed Technology (2013) Vol. 27, pgs. 412-416).

The above-mentioned plants include strains to which two or more traits are imparted among abiotic stress tolerance, disease resistance, herbicide tolerance, pest resistance, a growth trait, a yield trait, nutrient uptake, product quality, a fertility trait, and the like as described above using a genetic engineering technique, a classical breeding technique, a genome breeding technique, a new breeding technique, a genome editing technique, or the like, and strains to which two or more of the properties of the parent strains are imparted by crossing plants having the same or different properties.

Examples of the commercially available plants to which tolerance to two or more herbicides are imparted that can be treated by compositions comprising a compound of formula (I) include the cotton “GlyTol™ LibertyLink™” and “GlyTol™ LibertyLink™” that are tolerant to glyphosate and glufosinate; the maize “Roundup Ready™ LibertyLink™ Maize” that is tolerant to glyphosate and glufosinate; the soybean “Enlist™ Soybean” that is tolerant to glufosinate and 2,4-D; the soybean “Genuity® Roundup Ready (trademark) 2 Xtend (trademark)” that is tolerant to glyphosate and dicamba; the maize and the soybean “OptimumGAT™” that are tolerant to glyphosate and an ALS inhibitor; the genetically modified soybeans “Enlist E3™” and “Enlist™ Roundup Ready® 2 Yield” that are tolerant to three herbicides of glyphosate, glufosinate, and 2,4-D; the genetically modified maize “Enlist™ Roundup Ready® Corn 2” that is tolerant to glyphosate, 2,4-D, and an aryloxyphenoxypropionate (FOPs) herbicide; the genetically modified maize “Enlist™ Roundup Ready® Corn 2” that is tolerant to glyphosate, 2,4-D, and an aryloxyphenoxypropionate (FOPs) herbicide; the genetically modified cotton “Bollgard II® XtendFlex™ Cotton” that is tolerant to dicamba, glyphosate, and glufosinate; and the genetically modified cotton “Enlist™ Cotton” that is tolerant to three herbicides of glyphosate, glufosinate, and 2,4-D. In addition, the cotton tolerant to glufosinate and 2,4-D, the cotton tolerant to both glufosinate and dicamba, the maize tolerant to both glyphosate and 2,4-D, the soybean tolerant to both glyphosate and an HPPD herbicide, and the genetically modified maize tolerant to glyphosate, glufosinate, 2,4-D, an aryloxyphenoxypropionate (FOPs) herbicide, and a cyclohexanedione (DIMs) herbicide have been also developed.

Examples of the commercially available plants to which herbicide tolerance and pest resistance are imparted that can be treated by compositions comprising a compound of formula (I) include the maize “YieldGard Roundup Ready®” and “YieldGard Roundup Ready® 2” that are tolerant to glyphosate and resistant to a corn borer; the maize “Agrisure® CB/LL” that is tolerant to glufosinate and resistant to a corn borer; the maize “Yield Gard® VT Root worm/RR2” that is tolerant to glyphosate and resistant to a corn rootworm; the maize “Yield Gard® VT Triple” that is tolerant to glyphosate and resistant to a corn rootworm and a corn borer; the maize “Herculex® I” that is tolerant to glufosinate and resistant to a lepidopteran maize pest (Cry1F) (for example, resistance to a western bean cutworm, a corn borer, a black cutworm, and a fall armyworm); the maize “YieldGard® Corn Rootworm/Roundup Ready® 2” that is tolerant to glyphosate and resistant to a corn rootworm; the maize “Agrisure® GT/RW” that is tolerant to glufosinate and resistant to a Coleoptera maize pest (Cry3A) (for example, resistant to a western corn rootworm, a northern corn rootworm, and a Mexican corn rootworm); the maize “Herculex® RW” that is tolerant to glufosinate and resistant to a Coleoptera maize pest (Cry34/35Abl) (for example, resistant to a western corn rootworm, a northern corn rootworm, and a Mexican corn rootworm); the maize “Yield Gard® VT Root worm/RR2” that is tolerant to glyphosate and resistant to a corn rootworm; and the cotton “Bollgard 3@XtendFlex®” that is tolerant to dicamba, glyphosate, and glufosinate and resistant to a lepidopteran cotton pest (for example, resistant to bollworms, a tobacco budworm, and armyworms).

The composition comprising a compound of formula (I) can be applied to a place where weeds are growing or likely to grow. Examples include a method of spraying the present composition on soil and a method of spraying the present composition on weeds.

In some variations, the application rate of a composition comprising a compound of formula (I) is generally 1 to 10,000 g per 10,000 m², 2 to 5,000 g per 10,000 m², 5 to 2,000 g per 10,000 m², 1 to 1000 g per 10,000 m², 1 to 500 g per 10,000 m², 1 to 100 g per 10,000 m², 1 to 75 g per 10,000 m², 15 to 1000 g per 10,000 m², 15 to 100 g per 10,000 m², 15 to 75 g per 10,000 m², or 15 to 60 g per 10,000 m², in terms of the total amount of the compound of formula (I), or a salt thereof (including an agriculturally suitable salt thereof).

In one variation, the application rate of a composition of the invention is generally 1 to 10,000 g per 10,000 m², 2 to 5,000 g per 10,000 m², 5 to 2,000 g per 10,000 m², 1 to 1000 g per 10,000 m², 1 to 500 g per 10,000 m², 1 to 100 g per 10,000 m², 1 to 75 g per 10,000 m², 15 to 1000 g per 10,000 m², 15 to 100 g per 10,000 m², 15 to 75 g per 10,000 m², or 15 to 60 g per 10,000 m², in terms of the total amount of a compound of formula (I) and the at least one compound selected from the group consisting of the herbicide compound group B and the safener group C.

In the present method, an adjuvant may be mixed in a composition of the invention, followed by application. The type of the adjuvant is not particularly limited, and examples of the adjuvant include oil-based adjuvants such as Agri-Dex® and methylated seed oil (MSO), non-ions (esters or ethers of polyoxyethylene) such as Induce, anions (substituted sulfonates) such as Gramine S, cations (polyoxyethylene amines) such as Genamin® T 200BM, and organic silicons such as Silwet® L77.

The pH and the hardness of the spray liquid prepared when a composition of the invention is applied are not particularly limited, and the pH is usually in the range of 5 to 9, and the hardness is usually in the range of 0 to 500.

The time period for applying a composition of the invention is not particularly limited, and is usually in the range of 5:00 AM to 9:00 PM, and the photon flux density is usually 10 to 2,500 mol/m²/s.

When a composition of the invention is applied to a crop field, it may be applied before sowing a crop seed, simultaneously with sowing a crop seed, and/or after sowing a crop seed. That is, the frequency of the application of a composition of the invention is once before, simultaneously with, or after sowing a crop seed, twice excluding before the sowing, excluding simultaneously with the sowing, or excluding after the sowing, or three times at all the timing.

When a composition of the invention is applied before sowing a crop seed, it is applied from 50 days before to immediately before the sowing, preferably from 30 days before to immediately before the sowing, more preferably from 20 days before to immediately before the sowing, and still more preferably from 10 days before to immediately before the sowing.

When a composition of the invention is applied after sowing a crop seed, it is usually applied from immediately after the sowing to before flowering. The composition is more preferably applied from immediately after the sowing to before the emergence, or from 1 to 6 leaf stages of the crop. The case where a composition of the invention is applied simultaneously with sowing a crop seed is the case where a sowing machine and a sprayer are integrated with each other.

In the step of applying a composition of the invention in a cultivation area, a compound of formula (I) or the compound and at least one additional compound selected from the group consisting of the herbicide compound group B and the safener group C are usually mixed with a carrier such as a solid carrier or a liquid carrier, and an auxiliary agent for formulation such as a surfactant is added if necessary to prepare a formulation. Preferable formulation types is aqueous liquid suspension formulations, oil-based suspension formulations, wettable powders, water dispersible granules, granules, water-based emulsions, oil-based emulsions, and emulsifiable concentrates, and more preferable formulation type is emulsifiable concentrates. Furthermore, a formulation containing a compound of formula I alone as an active ingredient and a formulation containing the at least one compound selected from the group consisting of the herbicide compound group B and the safener group C as an active ingredient may be used in combination. Furthermore, a formulation containing the present composition as active ingredients and a formulation containing another herbicide as an active ingredient may be used in combination.

Examples of the method of applying a composition of the invention in a cultivation area include a method of spraying it on the soil in the cultivation area and a method of spraying the present composition on a weeds that are growing. The composition is usually diluted with water, followed by spraying. The spray volume is not particularly limited, and is usually 50 to 1,000 L/ha, preferably 100 to 500 L/ha, and more preferably 140 to 300 L/ha.

Specific examples of the weed species to be controlled by the present composition include, but are not limited to, the weed species described below.

Urticaceae weeds to be controlled include Urtica urens.

Polygonaceae weeds to be controlled include Polygonum convolvulus, Polygonum lapathifolium, Polygonum pensylvanicum, Polygonum persicaria, Polygonum longisetum, Polygonum aviculare, Polygonum arenastrum, Polygonum cuspidatum, Rumex japonicus, Rumex crispus, Rumex obtusifolius, and Rumex acetosa.

Portulacaceae weeds to be controlled include Portulaca oleracea.

Caryophyllaceae weeds to be controlled include Stellaria media, Stellaria aquatica, Cerastium holosteoides, Cerastium glomeratum, Spergula arvensis, and Silene gallica.

Molluginaceae weeds to be controlled include Mollugo verticillate.

Chenopodiaceae weeds to be controlled include Chenopodium album, Chenopodium ambrosioides, Kochia scoparia, Salsola ka/i, and Atriplex spp.

Amaranthaceae weeds to be controlled include Amaranthus retroflexus, Amaranthus viridis, Amaranthus lividus, Amaranthus spinosus, Amaranthus hybridus, Amaranthus palmeri, Amaranthus patulus, Waterhemp (Amaranthus tuberculatus, Amaranthus rudis, or Amaranthus tamariscinus), Amaranthus blitoides, Amaranthus deflexus, Amaranthus quitensis, Alternanthera philoxeroides, Alternanthera sessilis, and Alternanthera tenella.

Papaveraceae weeds to be controlled include Papaver rhoeas, Papaver dubium, and Argemone Mexicana.

Brassicaceae weeds to be controlled include Raphanus raphanistrum, Raphanus sativus, Sinapis arvensis, Capsella bursa-pastoris, Brassica juncea, Brassica napus, Descurainia pinnata, Rorippa islandica, Rorippa sylvestris, Thlaspi arvense, Myagrum rugosum, Lepidium virginicum, and Coronopus didymus.

Capparaceae weeds to be controlled include Cleome affinis.

Fabaceae weeds to be controlled include Aeschynomene indica, Aeschynomene rudis, Sesbania exaltata, Cassia obtusifolia, Cassia occidentalis, Desmodium tortuosum, Desmodium adscendens, Desmodium illinoense, Trifolium repens, Pueraria lobata, Vicia angustifolia, Indigofera hirsuta, Indigofera truxillensis, and Vigna sinensis.

Oxalidaceae weeds to be controlled include Oxalis corniculata, Oxalis strica, and Oxalis oxyptera.

Geraniaceae weeds to be controlled include Geranium carolinense and Erodium cicutarium.

Euphorbiaceae weeds to be controlled include Euphorbia helioscopia, Euphorbia maculata, Euphorbia humistrata, Euphorbia esula, Euphorbia heterophylla, Euphorbia brasiliensis, Acalypha australis, Croton glandulosus, Croton lobatus, Phyllanthus corcovadensis, and Ricinus communis.

Malvaceae weeds to be controlled include Abutilon theophrasti, Sida rhombiforia, Sida cordifolia, Sida spinosa, Sida glaziovii, Sida santaremnensis, Hibiscus trionum, Anoda cristata, and Malvastrum coromandelianum.

Onagraceae weeds to be controlled include Ludwigia epilobioides, Ludwigia octovalvis, Ludwigia decurre, Oenothera biennis, and Oenothera laciniata.

Sterculiaceae weeds to be controlled include Waltheria indica.

Violaceae weeds to be controlled include Viola arvensis and Viola tricolor.

Cucurbitaceae weeds to be controlled include Sicyos angulatus, Echinocystis lobata, and Momordica charantia.

Lythraceae weeds to be controlled include Ammannia multiflora, Ammannia auriculata, Ammannia coccinea, Lythrum salicaria, and Rotala indica.

Elatinaceae weeds to be controlled include Elatine triandra and Elatine cahfornica.

Apiaceae weeds to be controlled include Oenanthe javanica, Daucus carota, and Conium maculatum.

Ceratophyllaceae weeds to be controlled include Ceratophyllum demersum.

Cabombaceae weeds to be controlled include Cabomba caroliniana.

Haloragaceae weeds to be controlled include Myriophyllum aquaticum, Myriophyllum verticillatum, Myriophyllum spicatum, and Myriophyllum heterophyllum.

Sapindaceae weeds to be controlled include Cardiospermum halicacabum.

Primulaceae weeds to be controlled include Anagallis arvensis.

Asclepiadaceae weeds to be controlled include Asclepias syriaca, and Ampelamus albidus.

Rubiaceae weeds to be controlled include Galium aparine, Galium spurium var. echinospermon, Spermacoce latifolia, Richardia brasiliensis, and Borreria alata.

Convolvulaceae weeds to be controlled include Ipomoea nil, Ipomoea hederacea, Ipomoea purpurea, Ipomoea hederacea var. integriuscula, Ipomoea lacunosa, Ipomoea triloba, Ipomoea acuminata, Ipomoea hederifolia, Ipomoea coccinea, Ipomoea quamoclit, Ipomoea grandifolia, Ipomoea aristolochiafolia, Ipomoea cairica, Convolvulus arvensis, Calystegia hederacea, Calystegia japonica, Merremia hedeacea, Merremia aegyptia, Merremia cissoides, and Jacquemontia tamnifolia.

Boraginaceae weeds to be controlled include Myosotis arvensis.

Lamiaceae weeds to be controlled include Lamium purpureum, Lamium amplexicaule, Leonotis nepetaefolia, Hyptis suaveolens, Hyptis lophanta, Leonurus sibiricus, and Stachys arvensis.

Solanaceae weeds to be controlled include Datura stramonium, Solanum nigrum, Solanum americanum, Solanum ptycanthum, Solanum sarrachoides, Solanum rostratum, Solanum aculeatissimum, Solanum sisymbriifolium, Solanum carolinense, Physalis angulata, Physalis subglabrata, and Nicandra physaloides.

Scrophulariaceae weeds to be controlled include Veronica hederaefolia, Veronica persica, Veronica arvensis, Lindernia procumbens, Lindernia dubia, Lindernia angustifolia, Bacopa rotundifolia, Dopatrium junceum, and Gratiola japonica.

Plantaginaceae weeds to be controlled include Plantago asiatica, Plantago lanceolata, Plantago major, and Callitriche palustris.

Asteraceae weeds to be controlled include Xanthium pensylvanicum, Xanthium occidentale, Xanthium italicum, Helianthus annuus, Matricaria chamomilla, Matricaria perforata, Chrysanthemum segetum, Matricaria matricarioides, Artemisia princeps, Artemisia vulgaris, Artemisia verlotorum, Solidago altissima, Taraxacum officinale, Galinsoga ciliata, Galinsoga parviflora, Senecio vulgaris, Senecio brasiliensis, Senecio grisebachii, Conyza bonariensis, Conyza smatrensis, Conyza canadensis, Ambrosia artemisiaefolia, Ambrosia trifida, Bidens tripartita, Bidens pilosa, Bidens frondosa, Bidens subalternans, Cirsium arvense, Cirsium vulgare, Silybum marianum, Carduus nutans, Lactuca serriola, Sonchus oleraceus, Sonchus asper, Wedelia glauca, Melampodium perfoliatum, Emilia sonchifolia, Tagetes minuta, Blainvillea latifolia, Tridax procumbens, Porophyllum ruderale, Acanthospermum australe, Acanthospermum hispidum, Cardiospermum halicacabum, Ageratum conyzoides, Eupatorium perfoliatum, Eclipta alba, Erechtites hieracifolia, Gamochaeta spicata, Gnaphalium spicatum, Jaegeria hirta, Parthenium hysterophorus, Siegesbeckia orientalis, Soliva sessilis, Eclipta prostrata, Eclipta alba, and Centipeda minima.

Alismataceae weeds to be controlled include Sagittaria pygmaea, Sagittaria trifolia, Sagittaria sagittifolia, Sagittaria montevidensis, Sagittaria aginashi, Alisma canaliculatum, and Alisma plantago-aquatica.

Limnocharitaceae weeds to be controlled include Limnocharis fiava.

Hydrocharitaceae weeds to be controlled include Limnobium spongia, Hydrilla verticillata, and Najas guadalupensis.

Araceae weeds to be controlled include Pistia stratiotes.

Lemnaceae weeds to be controlled include Lemna aoukikusa, Spirodela polyrhiza, and Wolffia spp.

Potamogetonaceae to be controlled include Potamogeton distinctus, Potamogeton crispus, Potamogeton illinoensis, and Stuckenia pectinata.

Liliaceae weeds to be controlled include Allium canadense, Allium vineale, and Allium macrostemon.

Pontederiaceae weeds to be controlled include Eichhornia crassipes, Heteranthera limosa, Monochoria korsakowii, and Monochoria vaginalis.

Commelinaceae weeds to be controlled include Commelina communis, Commelina bengharensis, Commelina erecta, and Murdannia keisak.

Poaceae weeds to be controlled include Echinochloa crus-galli, Echinochloa oryzicola, Echinochloa crus-galli var formosensis, Echinochloa oryzoides, Echinochloa colona, Echinochloa crus-pavonis, Setaria viridis, Setaria faberi, Setaria glauca, Setaria geniculata, Digitaria ciliaris, Digitaria sanguinalis, Digitaria horizontalis, Digitaria insularis, Eleusine indica, Poa annua, Poa trivialis, Poa pratensis, Alospecurus aequalis, Alopecurus myosuroides, Avena fatua, Sorghum halepense, Sorghum vulgare, Agropyron repens, Lolium multiflorum, Lolium perenne, Lolium rigidum, Bromus catharticus, Bromus sterilis, Bromus japonicus, Bromus secalinus, Bromus tectorum, Hordeum jubatum, Aegilops cylindrica, Phalaris arundinacea, Phalaris minor, Apera spica-venti, Panicum dichotomiforum, Panicum texanum, Panicum maximum, Brachiaria platyphylla, Brachiaria ruziziensis, Brachiaria plantaginea, Brachiaria decumbens, Brachiaria brizantha, Brachiaria humidicola, Cenchrus echinatus, Cenchrus pauciflorus, Eriochloa villosa, Pennisetum setosum, Chloris gayana, Chlorisvirgata, Eragrostis pilosa, Rhynchelitrum repens, Dactyloctenium aegyptium, Ischaemum rugosum, Isachne globosa, Oryza sativa, Paspalum notatum, Paspalum maritimum, Paspalum distichum, Pennisetum clandestinum, Pennisetum setosum, Rottboellia cochinchinensis, Leptochloa chinensis, Leptochloa fascicularis, Leptochloa fiiformis, Leptochloa panicoides, Leersia japonica, Leersia sayanuka, Leersia oryzoides, Glyceria leptorrhiza, Glyceria acutiflora, Glyceria maxima, Agrostis gigantea, Agrostis stolonifera, Cynodon dactylon, Dactylis glomerata, Eremochloa ophiuroides, Festuca arundinacea, Festuca rubra, Imperata cylindrica, Miscanthus sinensis, Panicum virgatum, and Zoysia japonica.

Cyperaceae weeds to be controlled include Cyperus microiria, Cyperus iria, Cyperus compressus, Cyperus difformis, Cyperus flaccidus, Cyperus globosus, Cyperus nipponics, Cyperus odoratus, Cyperus serotinus, Cyperus rotundus, Cyperus esculentus, Kyllinga gracillima, Kyllinga brevifolia, Fimbristylis miliacea, Fimbristylis dichotoma, Eleocharis acicularis, Eleocharis kuroguwai, Schoenoplectiella hotarui, Schoenoplectiella juncoides, Schoenoplectiella wallichii, Schoenoplectiella mucronatus, Schoenoplectiella triangulatus, Schoenoplectiella nipponicus, Schoenoplectiella triqueter, Bolboschoenus koshevnikovii, and Bolboschoenus fluviatilis.

Equisetaceae weeds to be controlled include Equisetum arvense, and Equisetum palustre.

Salviniaceae weeds to be controlled include Salvinia natans.

Azollaceae weeds to be controlled include Azolla japonica and Azolla imbricata. Marsileaceae weeds to be controlled include Marsilea quadrifolia.

Other weeds to be controlled include Pithophora, Cladophora, Bryophyta, Marchantiophyta, Anthocerotophyta, Cyanobacteria, Pteridophyta, sucker of perennial crops (pomaceous fruits, nut trees, citruses, Humulus lupulus, grapes, and the like).

In the above-mentioned weeds to be controlled, mutations within the species are not particularly limited. That is, the weeds include weeds having reduced sensitivity to a specific herbicide. The reduced sensitivity may be attributed to a mutation at a target site (target site mutation) or may be attributed to any factors other than the target site mutation (non-target site mutation). Examples of the factor of the reduced sensitivity due to a non-target site mutation include increased metabolism, malabsorption, translocation dysfunction, and excretion to out of system. Examples of the factor of the increased metabolism include the enhanced activity of a metabolizing enzyme such as cytochrome P450 monooxygenase, aryl acylamidase, esterase, or glutathione S-transferase. Examples of the excretion to out of system include transport to the vacuole by an ABC transporter. Examples of the weeds having reduced sensitivity due to a target site mutation include weeds having any one of or two or more of the following amino acid substitutions in the ALS gene: Ala122Thr, Ala122Val, Ala122Tyr, Pro197Ser, Pro197His, Pro197Thr, Pro197Arg, Pro197Leu, Pro197Gln, Pro197Ala, Pro197Ile, Ala205Val, Ala205Phe, Asp376Glu, Arg377His, Trp574Leu, Trp574Gly, Trp574Met, Ser653Thr, Ser653Thr, Ser653Asn, Ser635Ile, Gly654Glu, and Gly645Asp. Similarly, examples of the weeds having reduced sensitivity due to a target site mutation include weeds having any one of or two or more of the following amino acid substitutions in the ACCase gene: Ile1781Leu, Ile1781Val, Ile1781Thr, Trp1999Cys, Trp1999Leu, Ala2004Val, Trp2027Cys, Ile204lAsn, Ile2041Val, Asp2078Gly, Cys2088Arg, Gly2096Ala, and Gly2096Ser.

Similarly, as an example of the weeds having reduced sensitivity due to a target site mutation, PPO inhibitor-resistant weeds having one or more mutations selected from an Arg128Leu mutation, an Arg128Met mutation, an Arg128Gly mutation, an Arg128His mutation, a Gly210 deletion mutation, and a Gly399Ala mutation in PPO. The word “PPO” means protoporphyrinogen oxidase. Weeds usually have PPO1 and PPO2 in PPO, and the above-mentioned mutations may be present in either PPO1 or PPO2 or in both. The case where weeds have the mutations in PPO2 is preferable. For example, the word “Arg128Met” means that the mutation is present in the 128th (the number is standardized with PPO2 of Amaranthus palmeri) amino acid. In PPO2 of Ambrosia artemisiaefolia, the mutation corresponds to a mutation in the 98th amino acid (Rousonelos, et al., Weed Science (2012) Vol. 60, pgs. 335-344) and is known as Arg98Leu. In this case, Arg98 is equivalent to Arg128 according to the present invention. The Arg128Met mutation and the Arg128Gly mutation in the PPO of the weed to be controlled in the present invention are known in Amaranthus palmeri (Giacomini, et al., Pest Management Science (2017) Vol. 73, pgs. 1559-1563), the Arg128His mutation is known in Lolium rigidum (Fernandez-Moreno, et al., Weed Science Society of America (WSSA) annual meeting, 2018), and the Gly399Ala mutation is known in Amaranthus palmeri (Rangani, et al., WSSA annual meeting, 2018). In the present invention, the above-mentioned reported resistant weeds are particularly effectively controlled, but particularly effectively controlled weeds are not limited thereto. That is, other weeds having the amino acid mutation are similarly controlled. Not only Amaranthus palmeri having an Arg128Leu mutation, an Arg128Met mutation, an Arg128Gly mutation, an Arg128His mutation, a Gly210 deletion mutation, or a Gly399Ala mutation, but also, for example, waterhemp having the above-mentioned mutation, Ambrosia artemisiaefolia having the above-mentioned mutation, Lolium rigidum having the above-mentioned mutation, Lolium multiflorum having the above-mentioned mutation, and Euphorbia heterophylla having the above-mentioned mutation are effectively controlled.

Similarly, examples of the weeds having reduced sensitivity due to a target site mutation include weeds having an amino acid substitution such as Thr102Ile, Pro106Ser, Pro106Ala, or Pro106Leu in the EPSP gene. In particular, Eleusine indica, Lolium multiflorum, Lolium rigidum, Digitaria insularis, waterhemp, Echinochloa colona, and the like which are resistant to glyphosate and have one or both of the mutations are effectively controlled. Similarly, examples of the weeds having reduced sensitivity due to a target site include weeds having increased copies of the EPSP gene and Amaranthus palmeri, waterhemp, Kochia scoparia, and the like which are resistant to glyphosate and have the mutation are particularly effectively controlled. Conyza canadensis, Conyza smatrensis, and Conyza bonariensis which are resistant to glyphosate in which an ABC transporter is involved are also effectively controlled.

In the cultivation of a crop according to the present invention, plant nutritional management in general cultivation of a crop can be performed. The fertilization system may be based on Precision Agriculture or may be conventionally uniform one. In addition, a nitrogen-fixing bacterium or a mycorrhizal fungus can be inoculated in combination with seed treatment.

### Preparation of Compounds of Formula (I)

In one general example, a compound of formula (I) can be prepared as shown in Scheme 1a.

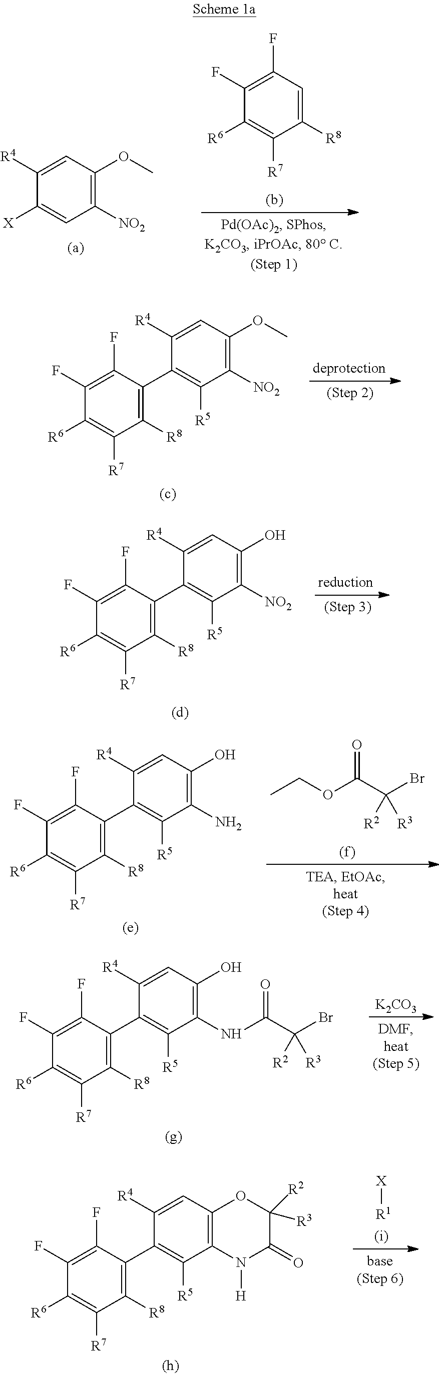

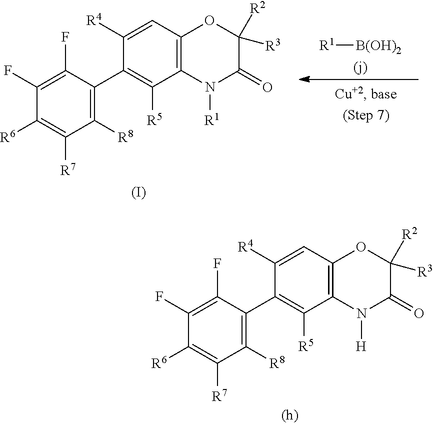

Accordingly, compounds of formula c can be prepared by reaction of a compound of formula (a), where X is Br or I, with a substituted phenyl of formula (b) using cross-coupling reaction conditions with the aid of a metal catalyst as shown in Step 1 of Scheme 1a. Suitable catalysts include palladium catalysts, such as Pd(OAc)₂ combined with 2-dicyclohexylphosphino-2′,6′-dimethoxybiphenyl (SPhos). As shown in Step 2 of Scheme 1a, compounds of formula (d) can be prepared by demethylation of the aryl methyl ether of a compound of formula (c) under acidic conditions. In one example, a Lewis acid such as boron tribromide can be used. As shown in Step 3 of Scheme 1a, compounds of formula (e) can be prepared by reduction of the nitro group of a compound of formula (d). Several methods for this are known to those skilled in the art, including the use of catalytic hydrogenation, sodium sulfide, or sodium hydrosulfite. As shown in Step 4 of Scheme 1a, compounds of formula (g) can be prepared by condensing the amino group of a compound of formula (e) with a suitable haloacetate of formula (f) under basic conditions in an organic solvent. In one example, the base is a trialkylamine such as triethylamine or diisopropylethylamine. As shown in Step 5 of Scheme 1a, benzoxazinones of formula (h) (a compound of formula (I), wherein R¹ is H) can be prepared via intramolecular ring closure between the phenolic hydroxyl group and N-acyl halide of a compound of formula (g) in a suitable polar organic solvent such as DMF or DMSO. As shown in Step 6 of Scheme 1a, a compound of formula (I) (wherein R¹ is, for example, an optionally substituted C₁₋₄alkyl) can be formed by reacting the benzoxazinone amino group of a compound of formula (h) with an alkyl or aryl halide of formula (i) under conditions suitable for bond formation. Alternatively, a compound of formula (h) can be reacted with a boronic acid of formula (j) using a Chan-Lam type coupling to form a compound of formula (I).

In another general example, a compound of formula (I) can be prepared as shown in Scheme 1b.

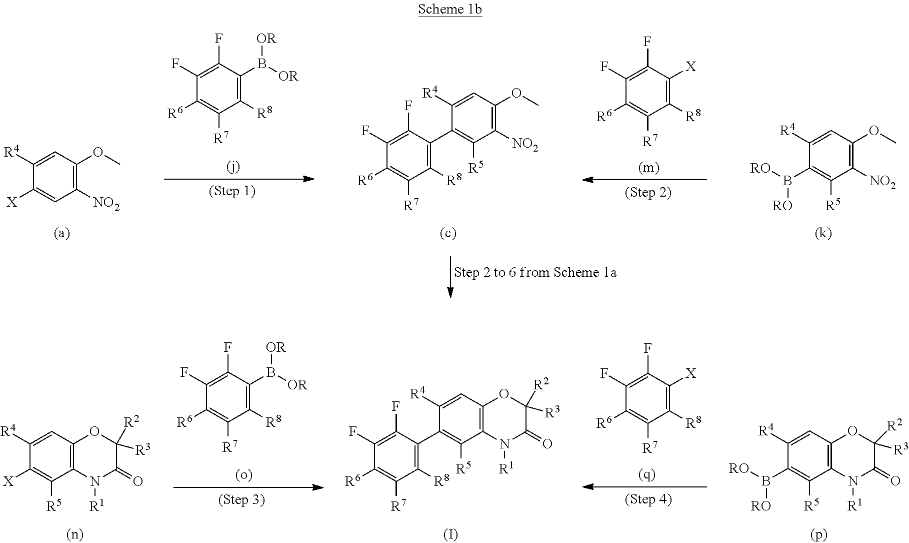

Accordingly, phenyl boronic acids (where R═H) or phenyl boronates (e.g., where —B(OR)₂ represents a pinacol ester) of formula (j) can be coupled to a suitably substituted phenyl bromide or iodide in a Suzuki-Miyaura-type reaction using a suitable catalyst to produce a compound of formula (c) (Step 1). This can also be accomplished under similar conditions by reacting a compound for formula (k) with a compound (m) (Step 2). Employing steps analogous to Steps 2 to 6 as described in Scheme 1a can then be used to transform a compound of formula (c) to a compound of formula (I). Alternatively, a compound of formula (n) can be reacted with a compound of formula (o) (Step 3) or a compound of formula (p) can be reacted with a compound of formula (q) (Step 4) under Suzuki conditions to produce a compound of formula (I).

### Formulation

In certain aspects, a composition of this disclosure or at least one part of a composition described herein, including those including agriculturally suitable salts thereof, may include at least one additional component selected from the group consisting of surfactants, solid diluents, and liquid diluents, which serves as a carrier. The formulation ingredients are selected to be consistent with the physical properties of the active ingredient, mode of application, and environmental factors such as soil type, moisture, and temperature.

Liquid formulations include solutions (including emulsifiable concentrates), suspensions, emulsions (including microemulsions, oil-in-water emulsions, flowable concentrates and/or suspoemulsions), and the like, which optionally can be thickened into gels. The general types of aqueous liquid formulations are soluble concentrate, suspension concentrate, capsule suspension, concentrated emulsion, microemulsion, oil-in-water emulsion, flowable concentrate, and suspoemulsion. The general types of nonaqueous liquid formulations are emulsifiable concentrate, microemulsifiable concentrate, dispersible concentrate, and oil dispersion.

The general types of solid formulations are dusts, powders, granules, pellets, prills, pastilles, tablets, filled films (including seed coatings), and the like, which can be water-dispersible (“wettable”) or water-soluble. Films and coatings formed from film-forming solutions or flowable suspensions are particularly useful for seed treatment. Active ingredient can be (micro)encapsulated and further formed into a suspension or solid formulation. Alternatively, the entire formulation of active ingredient can be encapsulated (or “overcoated”). Encapsulation can control or delay release of the active ingredient. An emulsifiable granule combines the advantages of both an emulsifiable concentrate formulation and a dry granular formulation. High-strength formulations are primarily used as intermediates for further formulation.

Sprayable formulations are typically extended in a suitable medium before spraying. Such liquid and solid formulations are formulated to be readily diluted in the spray medium, usually water, but occasionally another suitable medium like an aromatic or paraffinic hydrocarbon or vegetable oil. Spray volumes can range from about from about one to several thousand liters per hectare, but more typically are in the range from about ten to several hundred liters per hectare. Sprayable formulations can be tank mixed with water or another suitable medium for foliar treatment by aerial or ground application, or for application to the growing medium of the plant.

Liquid and dry formulations can be metered directly into drip irrigation systems or metered into the furrow during planting.

The formulations will typically contain effective amounts of active ingredient, diluent, and surfactant within the following approximate ranges, shown in Table 2, which add up to 100 percent by weight.

TABLE 2
Formulation Ratios

| | Weight Percent | | |
|---|---|---|---|
| | Active | | |
| | Ingredient | Diluent | Surfactant |
| Water-Dispersible and Water- | 0.001-90 | 0-99.999 | 0-15 |
| Soluble Granules, Tablets, and | | | |
| Powders | | | |
| Oil Dispersions, Suspensions, | 1-50 | 40-99 | 0-50 |
| Emulsions Solutions (including | | | |
| emulsifiable Concentrates) | | | |
| Dusts | 1-25 | 70-99 | 0-5 |
| Granules and Pellets | 0.001-99 | 5-99.999 | 0-15 |
| High Strength Formulations | 90-99 | 0-10 | 0-2 |

Solid diluents include, for example, clays such as bentonite, montmorillonite, attapulgite and kaolin, gypsum, cellulose, titanium dioxide, zinc oxide, starch, dextrin, sugars (e.g., lactose, sucrose), silica, talc, mica, diatomaceous earth, urea, calcium carbonate, sodium carbonate and bicarbonate, and sodium sulfate. Typical solid diluents are described in Watkins et al., Handbook of Insecticide Dust Diluents and Carriers, 2nd Ed., Dorland Books, Caldwell, New Jersey.

Liquid diluents include, for example, water; N,N-dimethylalkanamides (e.g., N,N-dimethylformamide); limonene; dimethyl sulfoxide; N-alkylpyrrolidones (e.g., N-methylpyrrolidinone); alkyl phosphates (e.g., triethyl phosphate); ethylene glycol; triethylene glycol; propylene glycol; dipropylene glycol; polypropylene glycol; propylene carbonate; butylene carbonate; paraffins (e.g., white mineral oils, normal paraffins, isoparaffins); alkylbenzenes; alkylnaphthalenes; glycerine; glycerol triacetate; sorbitol; aromatic hydrocarbons; dearomatized aliphatics; alkylbenzenes; alkylnaphthalenes; ketones such as cyclohexanone, 2-heptanone, isophorone, and 4-hydroxy-4-methyl-2-pentanone; acetates such as isoamyl acetate, hexyl acetate, heptyl acetate, octyl acetate, nonyl acetate, tridecyl acetate, and isobornyl acetate; other esters such as alkylated lactate esters, dibasic esters, alkyl and aryl benzoates, and γ-butyrolactone; and alcohols, which can be linear, branched, saturated or unsaturated, such as methanol, ethanol, n-propanol, isopropyl alcohol, n-butanol, isobutyl alcohol, n-hexanol, 2-ethylhexanol, n-octanol, decanol, isodecyl alcohol, isooctadecanol, cetyl alcohol, lauryl alcohol, tridecyl alcohol, oleyl alcohol, cyclohexanol, tetrahydrofurfuryl alcohol, diacetone alcohol, cresol, and benzyl alcohol. Liquid diluents also include glycerol esters of saturated and unsaturated fatty acids (typically C₆-C₂₂) such as plant seed and fruit oils (e.g., oils of olive, castor, linseed, sesame, corn (maize), peanut, sunflower, grapeseed, safflower, cottonseed, soybean, rapeseed, coconut, and palm kernel), animal-sourced fats (e.g., beef tallow, pork tallow, lard, cod liver oil, fish oil), and mixtures thereof. Liquid diluents also include alkylated fatty acids (e.g., methylated, ethylated, butylated) wherein the fatty acids may be obtained by hydrolysis of glycerol esters from plant and animal sources and can be purified by distillation. Typical liquid diluents are described in C. Marsden & S. Mann, Solvents Guide, Cleaver-Hume Press, London, 1963.

Surfactants can be classified as nonionic, anionic, or cationic. Nonionic surfactants useful for the present formulations include, but are not limited to: alcohol alkoxylates such as alcohol alkoxylates based on natural and synthetic alcohols (which may be branched or linear) and prepared from the alcohols and ethylene oxide, propylene oxide, butylene oxide or mixtures thereof; amine ethoxylates, alkanolamides, and ethoxylated alkanolamides; alkoxylated triglycerides such as ethoxylated soybean, castor, and rapeseed oils; alkylphenol alkoxylates such as octylphenol ethoxylates, nonylphenol ethoxylates, dinonyl phenol ethoxylates, and dodecyl phenol ethoxylates (prepared from the phenols and ethylene oxide, propylene oxide, butylene oxide or mixtures thereof); block polymers prepared from ethylene oxide or propylene oxide and reverse block polymers where the terminal blocks are prepared from propylene oxide; ethoxylated fatty acids; ethoxylated fatty esters and oils; ethoxylated methyl esters; ethoxylated tristyrylphenol (including those prepared from ethylene oxide, propylene oxide, butylene oxide, or mixtures thereof); fatty acid esters, glycerol esters, lanolin-based derivatives, polyethoxylate esters such as polyethoxylated sorbitan fatty acid esters, polyethoxylated sorbitol fatty acid esters, and polyethoxylated glycerol fatty acid esters; other sorbitan derivatives such as sorbitan esters; polymeric surfactants such as random copolymers, block copolymers, alkyd PEG (polyethylene glycol) resins, graft or comb polymers and star polymers; polyethylene glycols (PEGs); polyethylene glycol fatty acid esters; silicone-based surfactants; and sugar-derivatives such as sucrose esters, alkyl polyglycosides, and alkyl polysaccharides.

Useful anionic surfactants include, but are not limited to: alkylaryl sulfonic acids and their salts; carboxylated alcohol or alkylphenol ethoxylates; diphenyl sulfonate derivatives; lignin and lignin derivatives such as lignosulfonates; maleic or succinic acids or their anhydrides; olefin sulfonates; phosphate esters such as phosphate esters of alcohol alkoxylates, phosphate esters of alkylphenol alkoxylates and phosphate esters of styryl phenol ethoxylates; protein-based surfactants; sarcosine derivatives; styryl phenol ether sulfate; sulfates and sulfonates of oils and fatty acids; sulfates and sulfonates of ethoxylated alkylphenols; sulfates of alcohols; sulfates of ethoxylated alcohols; sulfonates of amines and amides such as N,N-alkyltaurates; sulfonates of benzene, cumene, toluene, xylene, and dodecyl and tridecylbenzenes; sulfonates of condensed naphthalenes; sulfonates of naphthalene and alkyl naphthalene; sulfonates of fractionated petroleum; sulfosuccinamates; and sulfosuccinates and their derivatives such as dialkyl sulfosuccinate salts.

Useful cationic surfactants include, but are not limited to: amides and ethoxylated amides; amines such as N-alkyl propanediamines, tripropylenetriamines, and dipropylenetetramines, and ethoxylated amines, ethoxylated diamines and propoxylated amines (prepared from the amines and ethylene oxide, propylene oxide, butylene oxide or mixtures thereof); amine salts such as amine acetates and diamine salts; quaternary ammonium salts such as quaternary salts, ethoxylated quaternary salts, and diquaternary salts; and amine oxides such as alkyldimethylamine oxides and bis-(2-hydroxyethyl)-alkylamine oxides.

Also useful for the present formulations are mixtures of nonionic and anionic surfactants or mixtures of nonionic and cationic surfactants. Nonionic, anionic, and cationic surfactants and their recommended uses are disclosed in a variety of published references including McCutcheon's Emulsifiers and Detergents, annual American and International Editions published by McCutcheon's Division, The Manufacturing Confectioner Publishing Co.; Sisely and Wood, Encyclopedia of Surface Active Agents, Chemical Publ. Co., Inc., New York, 1964; and A. S. Davidson and B. Milwidsky, Synthetic Detergents, Seventh Edition, John Wiley and Sons, New York, 1987.

Formulations of the present invention may also contain formulation auxiliaries and additives, known to those skilled in the art as formulation aids (some of which may be considered to also function as solid diluents, liquid diluents, or surfactants). Such formulation auxiliaries and additives may control the following: pH (buffers), foaming during processing (antifoams such polyorganosiloxanes), sedimentation of active ingredients (suspending agents), viscosity (thixotropic thickeners), in-container microbial growth (antimicrobials), product freezing (antifreezes), color (dyes/pigment dispersions), wash-off (film formers or stickers), evaporation (evaporation retardants), and other formulation attributes. Film formers include, for example, polyvinyl acetates, polyvinyl acetate copolymers, polyvinylpyrrolidone-vinyl acetate copolymer, polyvinyl alcohols, polyvinyl alcohol copolymers, and waxes. Examples of formulation auxiliaries and additives include those listed in McCutcheon's Volume 2: Functional Materials, annual International and North American editions published by McCutcheon's Division, The Manufacturing Confectioner Publishing Co.; and PCT Publication WO 03/024222.

The compounds of the invention and any other active ingredients are typically incorporated into the present formulations by dissolving the active ingredient in a solvent or by grinding in a liquid or dry diluent. Solutions, including emulsifiable concentrates, can be prepared by simply mixing the ingredients. If the solvent of a liquid formulations intended for use as an emulsifiable concentrate is water-immiscible, an emulsifier is typically added to emulsify the active-containing solvent upon dilution with water. Active ingredient slurries, with particle diameters of up to 2,000 microns can be wet milled using media mills to obtain particles with average diameters below 3 microns. Aqueous slurries can be made into finished suspension concentrates (see, for example, U.S. Pat. No. 3,060,084) or further processed by spray drying to form water-dispersible granules. Dry formulations usually require dry milling processes, which produce average particle diameters in the 2 micron to 10 micron range. Dusts and powders can be prepared by blending and usually grinding (such as with a hammer mill or fluid-energy mill). Granules and pellets can be prepared by spraying the active material upon preformed granular carriers or by agglomeration techniques. See Browning, “Agglomeration,” Chemical Engineering, Dec. 4, 1967, pp 147-48, Perry's Chemical Engineer's Handbook, 4th Ed., McGraw-Hill, New York, 1963, pages 8-57 and following, and PCT Publication WO 91/13546. Pellets can be prepared as described in U.S. Pat. No. 4,172,714. Water-dispersible and water-soluble granules can be prepared as taught in U.S. Pat. Nos. 4,144,050 and 3,920,442 and German Pat. No. 3,246,493. Tablets can be prepared as taught in U.S. Pat. Nos. 5,180,587, 5,232,701, and 5,208,030. Films can be prepared as taught in Great Britain Pat. No. 2,095,558 and U.S. Pat. No. 3,299,566.

For further information regarding the art of formulation, see T. S. Woods, “The Formulator's Toolbox—Product Forms for Modern Agriculture” in Pesticide Chemistry and Bioscience, The Food-Environment Challenge, T. Brooks and T. R. Roberts, Eds., Proceedings of the 9th International Congress on Pesticide Chemistry, The Royal Society of Chemistry, Cambridge, 1999, pp. 120-133. See also U.S. Pat. No. 3,235,361, Col. 6, line 16 through Col. 7, line 19 and Examples 10-41; U.S. Pat. No. 3,309,192, Col. 5, line 43 through Col. 7, line 62 and Examples 8, 12, 15, 39, 41, 52, 53, 58, 132, 138-140, 162-164, 166, 167 and 169-182; U.S. Pat. No. 2,891,855, Col. 3, line 66 through Col. 5, line 17 and Examples 1-4; Klingman, Weed Control as a Science, John Wiley and Sons, Inc., New York, 1961, pp 81-96; Hance et al., Weed Control Handbook, 8th Ed., Blackwell Scientific Publications, Oxford, 1989; and Developments informulation technology, PJB Publications, Richmond, U K, 2000.

### EXAMPLES

The presently disclosed subject matter will be better understood by reference to the following examples, which are provided as exemplary of the invention, and not by way of limitation.

The expected response value (E) of mortality or growth inhibition is determined using Colby's equation E=(X+Y)−(XY)/100 for each herbicide combination (Colby, Calculating Synergistic and Antagonistic Responses of Herbicide Combinations. Weeds (1967) Vol. 15, pgs. 20-22), where X is the percent weed control by a compound of formula (I) and Y is the percent weed control by a herbicide from Group B. The effect of the combination is synergistic when the observed response is greater than E.

### Example 1. Weed Control when Various Weeds are Sown and Pyroxasulfone-Containing Combinations are Applied to the Soil 7 Days Before Soybean Seeding

Weeds (Amaranthus palmeri, waterhemp, Ambrosia artemisiaefolia, Ambrosia trifida, Conyza canadensis, Chenopodium album, Kochia scoparia, Echinochloa crusgalli, and Setaria faberi) are sown in a plastic pot. On the same day, a mixed spray liquid combination of the compound of formula (I) and ZIDUA is applied on the soil surface at a spray volume of 200 L/ha. The application rate of the compound of formula (I) may be 25, 50, 100, or 200 g/ha and the application rate of ZIDUA (pyroxasulfone 85% water dispersible granule, manufactured by BASF SE) may be 70, 140, or 280 g/ha (1, 2, or 4 ounces/acre). The weeds are then cultivated in a greenhouse, and at 7 days after the application, soybeans are sown. Fourteen days after seeding the soybeans, the effect on the weeds and the crop injury on the soybeans are investigated. A synergistic effect is observed when synergism by Colby's formula is satisfied.

### Example 2. Weed Control when Weeds are Sown and Saflufenacil-Containing Combinations are Applied to the Soil 7 Days Before Soybean Seeding

Weeds (Amaranthus palmeri, waterhemp, Ambrosia artemisiaefolia, Ambrosia trifida, Conyza canadensis, Chenopodium album, Kochia scoparia, Echinochloa crusgalli, and Setaria faberi) and soybeans are sown in a plastic pot. On the same day, a mixed spray liquid combination of the compound of formula (I) and Sharpen is applied on the soil surface at a spray volume of 200 L/ha. The application rate of the compound of formula (I) may be 25, 50, 100, or 200 g/ha and the application rate of Sharpen (saflufenacil 29.7% wettable powder, manufactured by BASF SE) may be 73 ml/ha (1 fluid ounce/acre). Then the weeds and the soybeans are cultivated in a greenhouse, and 21 days after the application, the effect on the weeds and the crop injury on the soybeans are investigated. A synergistic effect is observed when synergism by Colby's formula is satisfied.

### Example 3. Weed Control when Weeds and Soybean are Sown and Flumioxazin-Containing Combinations are Applied to the Soil on the Same Day as Weed and Soybean Seeding

Weeds (Amaranthus palmeri, waterhemp, Ambrosia artemisiaefolia, Ambrosia trifida, Conyza canadensis, Chenopodium album, Kochia scoparia, Echinochloa crusgalli, and Setaria faberi) and soybeans are sown in a plastic pot. On the same day, a mixed spray liquid combination of the compound of formula (I) and Valor SX is applied on the soil surface at a spray volume of 200 L/ha. The application rate of the compound of formula (I) may be 25, 50, 100, or 200 g/ha and the application rate of Valor SX (flumioxazin 51% wettable powder, manufactured by Valent U.S.A. LLC) may be 140 g/ha (2 ounces/acre). The weeds and soybeans are cultivated in a greenhouse and, 21 days after the application, the effect on the weeds and the crop injury on the soybeans are investigated. A synergistic effect is observed when synergism by Colby's formula is satisfied.

### Examples 4 to 6

Tests are carried out in the same manner as in Examples 1 to 3 except that the soybeans are replaced with corns or cottons.

### Example 7. Weed Control when Various Glyphosate-Containing Combinations are Applied to Foliage 21 Days after Weed Seeding

Weeds (Amaranthus palmeri, waterhemp, Ambrosia artemisiaefolia, Ambrosia trifida, Conyza canadensis, Chenopodium album, Kochia scoparia, Echinochloa crusgalli, and Setaria faberi) are sown in a plastic pot. Then the weeds are cultivated in a greenhouse and, 21 days after the sowing, a mixed spray liquid of a combination of the compound of formula (I) and Roundup is applied to the foliage at the spray volume of 200 L/ha. The application rate of the compound of formula (I) may be 25, 50, 100, or 200 g/ha and the application rate of RoundupWeatherMax (glyphosate-potassium salt 660 g/L, manufactured by Monsanto Company) may be 2.338 L/ha (32 fluid ounces/acre). The weeds are further cultivated in the greenhouse, and 7 days and 14 days after the treatment, the effect on the weeds is investigated. A synergistic effect is observed when synergism by Colby's formula is satisfied.

### Example 8. Weed Control when Weeds are Sown and a Compound of Formula (I) is Applied to Soil 7 Days Before Soybean Seeding and Pyroxasulfone Application

Weeds (Amaranthus palmeri, waterhemp, Ambrosia artemisiaefolia, Ambrosia trifida, Conyza canadensis, Chenopodium album, Kochia scoparia, Echinochloa crusgalli, and Setaria faberi) are sown in a plastic pot. On the same day, a spray liquid which is prepared so that the application rate of the compound of formula (I) is applied on the soil surface at the spray volume of 200 L/ha. The application rate may be 25, 50, 100, or 200 g/ha. Then the weeds are cultivated in a greenhouse, and 7 days after the application, soybeans are sown and a spray liquid of ZIDUA is applied at a spray volume of 200 L/ha. The application rate of ZIDUA (pyroxasulfone 85% water dispersible granule, manufactured by BASF SE) may be 70, 140, or 280 g/ha (1, 2, or 4 ounces/acre). Fourteen days after the sowing of soybeans, the effects on the weeds and the crop injury on the soybeans are investigated. A synergistic effect is observed when synergism by Colby's formula is satisfied.

### Example 9. Weed Control when Weeds are Sown and a Compound of Formula (I) is Applied to Soil 7 Days Before Soybean Seeding and Saflufenacil Application

Weeds (Amaranthus palmeri, waterhemp, Ambrosia artemisiaefolia, Ambrosia trifida, Conyza canadensis, Chenopodium album, Kochia scoparia, Echinochloa crusgalli, and Setaria faberi) are sown in a plastic pot. On the same day, a spray liquid prepared so that the application rate of the compound of formula (I) is applied on the soil surface at the spray volume of 200 L/ha. The application rate may be 25, 50, 100, or 200 g/ha. The weeds are then cultivated in a greenhouse and, 7 days after the application, soybeans are sown and a spray liquid of Sharpen is applied at the spray volume of 200 L/ha. Sharpen (saflufenacil 29.7% wettable powder, manufactured by BASF SE) may be 73 ml/ha (1 fluid ounce/acre). Fourteen days after the sowing of soybeans, the effects on the weeds and the crop injury on the soybeans are investigated. A synergistic effect is observed when synergism by Colby's formula is satisfied.

### Example 10. Weed Control when Weeds are Sown and a Compound of Formula (I) is Applied to Soil 7 Days Before Soybean Seeding and Flumioxazin Application

Weeds (Amaranthus palmeri, waterhemp, Ambrosia artemisiaefolia, Ambrosia trifida, Conyza canadensis, Chenopodium album, Kochia scoparia, Echinochloa crusgalli, and Setaria faberi) are sown in a plastic pot. On the same day, a spray liquid prepared so that the application rate of the compound of formula (I) is at a spray volume of 200 L/ha is applied on the soil surface. The application rate may be 25, 50, 100, or 200 g/ha. The weeds are cultivated in a greenhouse and, 7 days after the application, soybeans are sown and a spray liquid of Valor SX is applied at a volume of 200 L/ha. The application rate of Valor SX (flumioxazin 51% wettable powder, manufactured by Valent U.S.A. LLC) may be 140 g/ha (2 ounces/acre). Fourteen days after the sowing of soybeans, the effects on the weeds and the crop injury on the soybeans are investigated. A synergistic effect is observed when synergism by Colby's formula is satisfied.

### Examples 11 to 13

Tests are carried out in the same manner as in Examples 8 to 10 except that RoundupPowerMax (glyphosate-potassium salt 660 g/L, manufactured by Monsanto Company) is additionally used when the compound of formula (I) is applied such that the application rate of RoundupPowerMax may be 2.338 L/ha (32 fluid ounces/acre, 1,543 g/ha as glyphosate-potassium salt)

### Example 14. Weed Control when Weeds are Sown and a Compound of Formula (I) is Applied to Soil 7 Days Before Soybean Seeding and 14 Days Before Glyphosate Application

Weeds (Amaranthus palmeri, waterhemp, Ambrosia artemisiaefolia, Ambrosia trifida, Conyza canadensis, Chenopodium album, Kochia scoparia, Echinochloa crusgalli, and Setaria faberi) are sown in a plastic pot. On the same day, a spray liquid of the compound of formula (I) is applied on the soil surface at the spray volume of 200 L/ha. The compound of formula (I) may be prepared such that the application rate may be 25, 50, 100, or 200 g/ha. The weeds are then cultivated in a greenhouse and, 7 days after the application, soybeans are sown. Fourteen days after the initial sowing, a spray liquid of RoundupWeatherMax is applied at the spray volume of 200 L/ha. The application rate of RoundupWeatherMax (glyphosate-potassium salt 660 g/L, manufactured by Monsanto Company) may be 2.338 L/ha (32 fluid ounces/acre). Another 14 days later (28 days after the initial sowing), the effects on the weeds and the crop injury on the soybeans are investigated. A synergistic effect is observed when synergism by Colby's formula is satisfied.

### Example 15

A test is carried out in the same manner as in Example 14 except that RoundupPowerMax (glyphosate-potassium salt 660 g/L, manufactured by Monsanto Company) is additionally used in the application of the compound of formula (I) such that the application rate of RoundupPowerMax may be 2.338 L/ha (32 fluid ounces/acre, 1,543 g/ha as a glyphosate-potassium salt).

### Examples 16 to 23

Tests are carried out in the same manner as in Examples 8 to 15 except that the soybeans are replaced with corns or cottons.

In the case where any crops are used in Examples 1 to 23, tests are carried out in the same manner as in the Examples except that the crops used in the Examples are replaced with crops having a Roundup Ready 2 Xtend trait.

### Examples 24 to 46

In the case where any crops are used in Examples 1 to 23, tests are carried out in the same manner as in the Examples except that the crops used in the Examples are replaced with the crops having both a Roundup Ready 2 Xtend trait and a PPO inhibitor-tolerant trait due to foreign PPO having lower affinity for the PPO inhibitor than the endogenous PPO of the crops by genetic engineering.

### Examples 47 to 69

In the case where any crops are used in Examples 1 to 23, tests are carried out in the same manner as in the Examples except that the crops used in the Examples are replaced with the crops having both a Roundup Ready 2 Xtend trait and a PPO inhibitor-tolerant trait due to foreign PPO having lower affinity for the PPO inhibitor than the endogenous PPO of the crops by genetic engineering.

### Example 70. Field Testing of Compound 1—Glyphosate Mixtures

Compound 1 and Compound 1 mixed with glyphosate were tested at different field locations to evaluate the herbicides efficacy for controlling weeds by post-emergence foliar application. All herbicide treatments were applied with a backpack CO₂ pressurized sprayer at 190 L/ha volume. The applications were made after the weeds emerged and reached the 5 to 12 cm size range. Herbicide treatments included Compound 1 formulated as 10% emulsified concentrate, glyphosate as a commercial formulation (Roundup Power Max, Monsanto), or a mixture of Compound 1 and glyphosate products prepared as a “tank-mix” before the field application. All herbicide field spray solution included ammonium sulfate and concentrated crop oil adjuvant to a final solution concentration of 2% and 1% respectively. Different weeds species were assessed at the separate field locations. For each species, the herbicide efficacy to control weeds, assessed as % of dead plants relative to the untreated control plots, was evaluated at 2 weeks after the herbicide application to account for weed re-growth. The results as reported in Table 1 show the mixture of ENK-3171 and Glyphosate provided a better weed spectrum to control glyphosate resistant biotypes (e.g. AMAPA and AMATU at Loc 13 and Loc 15 respectively) and a wider specie type spectrum (e.g. AMATU and SETFA co-existing species at Loc 15).

TABLE 1

| | Loc 11 | | Loc 12 | Loc 13 | | Loc 14 | | Loc 15 | |
|---|---|---|---|---|---|---|---|---|---|
| Herbicide | (% dead) | | (% dead) | (% dead) | | (% dead) | | (% dead) | |
| treatment | AMATU | CHEAL | AMATU | AMAPA | ABUTH | AMATU | CHEAL | AMATU | SETFA |
| Compound 1 | 90 | 92 | 60 | 67 | 95 | 94 | 100 | 96 | 25 |
| 30 g a.i./ha | | | | | | | | | |
| Compound 1 | 97 | 98 | 67 | 91 | 100 | 96 | 100 | 98 | 28 |
| 60 g a.i./ha | | | | | | | | | |
| Compound 1 | 97 | 93 | 77 | 64 | 94 | 95 | 100 | 96 | 98 |
| 30 g a.i./ha + | | | | | | | | | |
| glyphosate | | | | | | | | | |
| 1120 g a.i./ha | | | | | | | | | |
| Compound 1 | 99 | 99 | 80 | 92 | 100 | 100 | 100 | 98 | 98 |
| 60 g a.i./ha + | | | | | | | | | |
| glyphosate | | | | | | | | | |
| 1120 g a.i./ha | | | | | | | | | |
| glyphosate | 88 | 88 | 50 | 13 | 85 | 32 | 88 | 12 | 100 |
| 1120 g a.i./ha | | | | | | | | | |

### Example 71. Field Testing of Compound 1—Pyroxasulfone Mixtures

Compound 1 and Compound 1 mixed with pyroxasulfone were tested at different field locations to evaluate the herbicides efficacy for controlling weeds by soil residual activity. All herbicide treatments were applied with a backpack CO₂ pressurized sprayer at 190 L/ha volume. The herbicide applications were made to bare soil free of any vegetation to assess the efficacy of the different herbicides to suppress weeds germination and development. The herbicide treatments included Compound 1 formulated as 10% emulsified concentrate, pyroxasulfone as a commercial formulation (ZIDUA SC, BASF), or a mixture of Compound 1 and pyroxasulfone products prepared as a “tank-mix” before the field application. All herbicide field spray solution included ammonium sulfate final solution concentration of 2%. Different weeds species were assessed at the separate field locations. For each species, the herbicide efficacy to control weeds, assessed as % of suppressed weeds plants relative to the untreated control plots, was evaluated at 6 weeks after the herbicide application. The results of the field tests reported in Table 2 show the mixture of ENK-3171 and pyroxasulfone provided a better weed spectrum and a clear significant improvement for weed control as mixtures of Compound 1 and pyroxasulfone provided a more consistent and robust herbicide efficacy across soil type, an improved weed control when different types of weeds are coexisting in the same field, and a longer lasting efficacy when compared to the single herbicide applications.

TABLE 2

| | Loc 1 | Loc 2 | Loc 3 | |
|---|---|---|---|---|
| Herbicide | (% growth inhibition) | (% growth inhibition) | (% growth inhibition) | |
| treatment | AMATU | AMATU | AMAPA | ABUTH |
| Compound 1 | 75 | 83 | 23 | 5 |
| (30 g a.i./ha) | | | | |
| Compound 1 | 88 | 95 | 63 | 25 |
| (60 g a.i./ha) | | | | |
| Compound 1 | 97 | 92 | 85 | 84 |
| (30 g a.i./ha) + | | | | |
| pyroxasulfone | | | | |
| (90 g a.i./ha) | | | | |
| Compound 1 | 99 | 98 | 89 | 85 |
| (60 g a.i./ha) + | | | | |
| pyroxasulfone | | | | |
| (90 g a.i./ha) | | | | |
| pyroxasulfone | 97 | 74 | 34 | 44 |
| (90 g a.i./ha) | | | | |

| | Loc 4 | | Loc 5 | | Loc 6 | |
|---|---|---|---|---|---|---|
| Herbicide | (% growth inhibition) | | (% growth inhibition) | | (% growth inhibition) | |
| treatment | ABUTH | SETFA | AMAPA | DIGSA | ABUTH | CHEAL |
| Compound 1 | 63 | 38 | 73 | 65 | 45 | 85 |
| (30 g a.i./ha) | | | | | | |
| Compound 1 | 65 | 55 | 85 | 78 | 60 | 95 |
| (60 g a.i./ha) | | | | | | |
| Compound 1 | 95 | 96 | 90 | 79 | 88 | 100 |
| (30 g a.i./ha) + | | | | | | |
| pyroxasulfone | | | | | | |
| (90 g a.i./ha) | | | | | | |
| Compound 1 | 94 | 96 | 93 | 95 | 85 | 100 |
| (60 g a.i./ha) + | | | | | | |
| pyroxasulfone | | | | | | |
| (90 g a.i./ha) | | | | | | |
| pyroxasulfone | 60 | 77 | 75 | 74 | 50 | 88 |
| (90 g a.i./ha) | | | | | | |

### Example 72. Post-Emergence Mixtures Including Compound 1

Compound 1 was tested at several doses for foliar weed control efficacy by itself or in combination with other herbicides. The following commercial herbicide were used in tank mix with a 10% active emulsifiable concentrate formulation of Compound 1: glyphosate (Roundup PowerMAX®, Bayer), mesotrione (Callisto®, Syngenta), 2,4-D dimethylamine salt (Weedar® 64, Nufarm), 2,4-D ester (Weedone® LV4, Nufarm), dicamba (Clash™, Nufarm), glufosinate (Liberty®, BASF).

The weed seeds were sown in 10″×20″ trays and propagated up to the transplanting stage which is 1-2 emerging leaves. One plant was then transplanted into 7×7×8 cm pots and sprayed with the compounds when plants reached the 3-4 true leaf stage for grass species or 10-15 cm for broadleaf species. The plants were propagated in a greenhouse with 26-28° C. day, 20-22° C. night temperature at 50-60%0 relative humidity and supplemental light using high pressure sodium grow lights when necessary. Three weeks after sowing, plants were sprayed with the test compound solution in a laboratory spray chamber fitted with 8003 even fan nozzle calibrated to deliver 187 L ha⁻¹ at 269 kPa. All treatments included the following inert ingredients in the final formulation; 100 Crop Oil Concentrate (v/v) (AGRI-DEX®, Helena Agri), 2.5% Ammonium sulphate (w/v), and 0.1% Tween-20 (v/v). Plants were evaluated for percentage visual growth inhibition compared to treated control, 14 days after treatment (DAT).

Post-emergence herbicidal activity of Compound 1 and other herbicides in tank mix or as individual applications on AMATU (Amaranthus rudis) and ECHCG (Echinochloa crus-gaili) are shown in Table 3.1 and Table 3.2

TABLE 3.1
Post-emergence percent weed growth inhibiton with Compound 1
mixed with other herbicides

| | | % Visual growth |
|---|---|---|
| | | inhibition |
| Treatment | Dose (g a.i./ha) | (AMATU) |
| Compound 1 | 30 | 66 |
| Compound 1 | 45 | 77 |
| Compound 1 | 60 | 84 |
| mesotrione | 105 | 61 |
| glufosinate | 450 | 96 |
| dicamba | 280 | 74 |
| 2,4-D acid | 280 | 89 |
| 2,4-D ester | 280 | 73 |
| glyphosate | 1000 | 60 |
| Compound 1 + glyphosate | 45 + 1000 | 80 |
| Compound 1 + glyphosate | 60 + 1000 | 100 |
| Compound 1 + mesotrione | 30 + 105 | 99 |
| Compound 1 + mesotrione | 45 + 105 | 98 |
| Compound 1 + mesotrione | 60 + 105 | 96 |
| Compound 1 + glufosinate | 30 + 450 | 97 |
| Compound 1 + glufosinate | 45 + 450 | 98 |
| Compound 1 + glufosinate | 60 + 450 | 98 |
| Compound 1 + dicamba | 30 + 280 | 89 |
| Compound 1 + dicamba | 45 + 280 | 93 |
| Compound 1 + dicamba | 60 + 280 | 98 |
| Compound 1 + 2,4-D acid | 30 + 280 | 98 |
| Compound 1 + 2,4-D acid | 45 + 280 | 99 |
| Compound 1 + 2,4-D acid | 60 + 280 | 99 |
| Compound 1 + 2,4-D ester | 30 + 280 | 94 |
| Compound 1 + 2,4-D ester | 45 + 280 | 96 |
| Compound 1 + 2,4-D ester | 60 + 280 | 98 |

TABLE 3.2
Post-emergence percent weed growth inhibiton with Compound 1
mixed with other herbicides

| | | % Visual growth |
|---|---|---|
| | | inhibition |
| Treatment | Dose (g a.i./ha) | (ECHCG) |
| Compound 1 | 45 | 56 |
| Compound 1 | 90 | 88 |
| Compound 1 | 120 | 82 |
| mesotrione | 105 | 95 |
| glufosinate | 450 | 41 |
| glyphosate | 860 | 84 |
| dicamba | 280 | 5 |
| 2,4-D acid | 280 | 10 |
| 2,4-D ester | 280 | 21 |
| Compound 1 + glyphosate | 45 + 860 | 65 |
| Compound 1 + glyphosate | 90 + 860 | 89 |
| Compound 1 + glyphosate | 120 + 860 | 100 |
| Compound 1 + mesotrione | 45 + 105 | 100 |
| Compound 1 + mesotrione | 90 + 105 | 100 |
| Compound 1 + mesotrione | 120 + 105 | 100 |
| Compound 1 + glufosinate | 45 + 450 | 91 |
| Compound 1 + glufosinate | 90 + 450 | 97 |
| Compound 1 + glufosinate | 120 + 450 | 93 |
| Compound 1 + dicamba | 45 + 280 | 26 |
| Compound 1 + dicamba | 90 + 280 | 26 |
| Compound 1 + dicamba | 120 + 280 | 69 |
| Compound 1 + 2,4-D acid | 45 + 280 | 52 |
| Compound 1 + 2,4-D acid | 90 + 280 | 68 |
| Compound 1 + 2,4-D acid | 120 + 280 | 78 |
| Compound 1 + 2,4-D ester | 45 + 280 | 61 |
| Compound 1 + 2,4-D ester | 90 + 280 | 88 |
| Compound 1 + 2,4-D ester | 120 + 280 | 88 |

### Example 73. Pre-Emergence Mixtures Including Compound 1

Compound 1 was tested at several doses for residual weed control efficacy by itself or in combination as a tank mix with commercial herbicides. The following commercial products were used in tank mix with a 10% active emulsifiable concentrate formulation of Compound 1: S-metolachlor (Dual Magnum®, Syngenta), mesotrione (Callisto®, Syngenta), pyroxasulfone (Zidua® SC, BASF), metribuzin (Tricor DF™, United Phosphorus, Inc).

Accordingly, weed seeds were planted in 2-inch pots containing custom field soil mix (sandy loam with 3.3% organic matter, pH 7.0) and covered with a fine layer of the same soil. Three replicate pots were treated with each compound. Treatment consisting of the above formulation excluding active compound was applied as a treatment control (TC). Pots were treated with the test compound solution in a laboratory spray chamber fitted with 8003 even fan nozzles calibrated to deliver 187-200 L ha⁻¹ at 269 kPa. All treatments included the following inert ingredients in the final formulation; 1% Crop Oil Concentrate (v/v) (AGRI-DEX, Helena Agri) and 2.5% Ammonium sulphate (w/v). Compound 1 was incorporated into the soil by simulating rainfall equivalent to 0.2 mm using the same track sprayer. Subsequently, pots were irrigated from the bottom until weed control evaluation. Pots were placed back in the greenhouse and evaluated for percentage visual growth and germination inhibition compared to TC 17-21 days after treatment (DAT). Complete growth inhibition is represented by 100%.

TABLE 4
Pre-emergence percent weed growth and germination inhibiton
with Compound 1 mixed with other herbicides.

| | Dose | | | | | | | |
|---|---|---|---|---|---|---|---|---|
| Treatment | (g a.i./ha) | AMAPA | AMATU | KCHSC | CHEAL | DIGSA | ALOMY | ECHCG |
| Compound 1 | 30 | 100% | 100% | 100% | 100% | 100% | 62% | 47% |
| Compound 1 | 60 | 100% | 100% | 100% | 100% | 100% | 100% | 85% |
| Compound 1 | 120 | 100% | 100% | 100% | 100% | 100% | 92% | 100% |
| S-metolachlor | 1000 | 65% | 80% | 57% | 50% | 80% | 30% | 93% |
| pyroxasulfone | 90 | 73% | 83% | 80% | 100% | 78% | 47% | 100% |
| mesotrione | 210 | 53% | 75% | 77% | 100% | 82% | 20% | 33% |
| metribuzin | 200 | 20% | 65% | 100% | 100% | 13% | 12% | 12% |
| Compound 1 + | 30 + 1000 | 100% | 100% | 100% | 100% | 100% | 100% | 100% |
| S-metolachlor | | | | | | | | |
| Compound 1 + | 15 + 90 | 100% | 100% | 100% | 100% | 100% | 88% | 100% |
| pyroxasulfone | | | | | | | | |
| Compound 1 + | 30 + 90 | 100% | 100% | 100% | 100% | 100% | 100% | 100% |
| pyroxasulfone | | | | | | | | |
| Compound 1 + | 30 + 210 | 100% | 100% | 100% | 100% | 100% | 57% | 100% |
| mesotrione | | | | | | | | |
| Compound 1 + | 30 + 200 | 100% | 100% | 100% | 100% | 100% | 75% | 92% |
| metribuzin | | | | | | | | |

1. A herbicidal composition including the compound of formula (I): 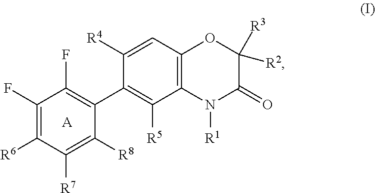
or a salt thereof, wherein:
R¹ is H or C₁₋₄alkyl optionally substituted with R^{1a}, phenyl, or benzyl, wherein each of said alkyl, phenyl or benzyl is optionally substituted with up to 3 F atoms, an OH group, or an OC₁₋₄alkyl group;
R^{1a} is 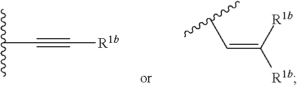
each R^{1b} is, independently, H, C₁₋₄alkyl, or cyclopropyl;
each of R² and R³ is, independently, H, Cl, F, CH₃, or R² and R³ together with the intervening carbon is cyclopropyl;
R⁴ is H, Cl, or F;
R⁵ is H or F;
each of R⁶ and R⁷ is, independently, F, H, C₁₋₂alkyl optionally substituted with OH, alkenyl, OH, OC₁₋₂alkyl, O-cyclopropyl, OCH₂CCH, NHCH₂Ph, N(R^{x})₂, or SCH₃;
R⁸ is H or F;
each R^{x} is, independently, H, CH₃, or C(O)CH₃; and
wherein Ring A contains at least 4 F atom substituents,
and at least one second herbicide selected from glyphosate, pyroxasulfone, mesotrione, glufosinate, dicamba, 2,4-D acid, 2,4-D ester, metribuzin, and S-metolachlor,
wherein a weight ratio of the compound of formula (I) to the second herbicide is about 1:0.1 to 1:50.

2. The herbicidal composition according to claim 1, wherein the compound of formula (I) is Compound 1:

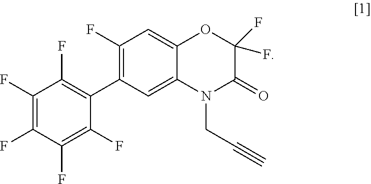 [SMILES: C#CCN1C(=O)C(F)(F)Oc2cc(F)c(-c3c(F)c(F)c(F)c(F)c3F)cc21]

3. The herbicidal composition according to claim 2, wherein the second herbicide is glyphosate and the weight ratio of Compound 1 to glyphosate is about 30:60 to 1000:1120.

4. The herbicidal composition according to claim 2, wherein the second herbicide is pyroxasulfone and the weight ratio of Compound 1 to pyroxasulfone is about 15:60 to 90.

5. The herbicidal composition according to claim 2, wherein the second herbicide is mesotrione and the weight ratio of Compound 1 to mesotrione is about 30:60 to 105:210.

6. The herbicidal composition according to claim 2, wherein the second herbicide is glufosinate and the weight ratio of Compound 1 to glufosinate is about 30:60 to 450.

7. The herbicidal composition according to claim 2, wherein the second herbicide is dicamba and the weight ratio of Compound 1 to dicamba is about 30:60 to 280.

8. The herbicidal composition according to claim 2, wherein the second herbicide is 2,4-D acid and the weight ratio of Compound 1 to 2,4-D acid is about 30:60 to 280.

9. The herbicidal composition according to claim 2, wherein the second herbicide is 2,4-D ester and the weight ratio of Compound 1 to 2,4-D ester is about 30:60 to 280.

10. The herbicidal composition according to claim 2, wherein the second herbicide is metribuzin and the weight ratio of Compound 1 to metribuzin is about 30 to 210.

11. The herbicidal composition according to claim 2, wherein the second herbicide is S-metolachlor and the weight ratio of Compound 1 to S-metolachlor is about 30 to 1000.

12. A method of controlling undesired vegetation, said method comprising contacting said vegetation or its environment with an herbicidally effective amount of the composition of claim 1.

13. The method according to claim 12, wherein the undesired vegetation comprises weeds.

14. The method according to claim 12, wherein the undesired vegetation comprises protoporphyrinogen IX oxidase (PPO) inhibitor-resistant weeds.

15. The method according to claim 14, wherein the PPO inhibitor-resistant weeds have a dG210 mutation.

16. The method according to claim 12, wherein contacting the undesired vegetation or its environment with the compound or composition leads to postemergence control of the undesired vegetation.

17. The method according to claim 12, wherein contacting the undesired vegetation or its environment with the compound or composition leads to preemergence control of the undesired vegetation.

18. The method according to claim 12, wherein the undesired vegetation is at least 60% controlled.
